(19) United States
(12) Patent Application Publication
(10) Pub. No.: US 2006/0025367 A1
(43) Pub. Date: Feb. 2, 2006

(54) Adenovirus vectors encoding brain natriuretic peptide

(75) Inventor: Robert D. Simari, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research, a Minnesota corporation

(21) Appl. No.: 11/185,327

(22) Filed: Jul. 20, 2005

Related U.S. Application Data
(62) Division of application No. 09/980,525, filed on Mar. 18, 2002.
(60) Provisional application No. 60/135,490, filed on May 24, 1999.

(51) Int. Cl. A61K 48/00

(52) U.S. Cl. 514/44

(57) ABSTRACT

The invention provides isolated and purified nucleic acid molecules encoding a natriuretic peptide useful in methods to inhibit or prevent heart failure.

### BACKGROUND OF THE INVENTION

Although major improvements in the treatment of cardiovascular disease have been achieved, many important limitations to current therapy exist. For example, heart failure remains a rapidly growing problem in the United States and the westem world, resulting in over 400,000 hospitalizations annually in the U.S. (Schocken et al., 1992). The focus on early left ventricular dysfunction is underscored by recent epidemiological evidence that at least 3% of the adult population above the age of 45 may have ventricular systolic dysfunction and 52% may be asymptomatic (McDonagh, 1997). The focus on early heart failure is also in response to the increasing emphasis of drug intervention in early left ventricular dysfunction. Such an emphasis is the result of clinical trials which have demonstrated improved mortality and morbidity with early treatment, although improvements have been modest (SOLVD investigators, 1991; Pfeffer et al., 1998).

The heart is an endocrine gland which plays a fundamental physiological role in the control of sodium homeostasis and arterial pressure via renal, myocardial, vascular and endocrine actions (DeBold et al., 1981; Stingo et al., 1992; Mattingly et al., 1994). Specifically, the heart serves as sensor of intravascular volume in which the cardiac atria senses increases in cardiac filling pressures and releases atrial natriuretic peptide (ANP) (Burnett et al., 1984). If cardiac volume or pressure overload is sustained, the atria and ventricles release brain natriuretic peptide (BNP). These two peptides function as circulating hormones to increase sodium excretion, promote vasodilatation and inhibit activation of the renin and endothelia systems (Koller et al., 1991; Stingo et al., 1998). These biological actions occur via the increase in the second messenger cGMP after peptide binding to a particulate guanylyl cyclase receptor (NPR-A receptor) (Fraenkel et al., 1994).

While ANP and BNP function in a redundant fashion, BNP has unique biological actions which include more potent natriuretic and lusitropic actions, more rapid gene expression, more potent inhibition of angiotensin II mediated ET gene activation, and a greater resistance to degradation by the ectoenzyrne neutral endopeptidase (NEP), as well as its superiority as a plasma marker for altered ventricular function and structure (Amin et al., 1995; Grantham et al., 1997; Bonow, 1996; Davidson et al., 1996). Other natriuretic peptides (NPs) include C-type natriuretic peptide (CNP), which is of endothelial cell origin, and D-type natriuretic peptide (DNP), which is of snake-origin (Schirger et al., 1999).

Natriuretic peptide particulate guanyl cyclase receptors are present in glomeruli and inner medullary collecting duct cells, as well as in vascular endothelial and smooth muscle cells and in cardiac myocytes and fibroblasts (Lopez et al., 1995; John et al., 1995; Steinhelper et al., 1990; Field et al., 1991). The use of antagonists to the natriuretic peptide particulate guanylyl cyclase receptors, as well as transgenic and gene knock-out studies, have supported a physiological role of the natriuretic peptide system (NPS) in cardiorenal regulation. Inhibition of the biologically active natriuretic peptide receptors (NPRs) in normal animals utilizing the receptor antagonist HS-142-1 have demonstrated reductions in sodium excretion, activation of renin release, impaired response to acute volume expansion, attenuation in the renal phenomenon of “DOCA escape,” reductions in coronary blood flow and an impairment in myocardial relaxation (Burnett et al., 1986; Cody et al., 1986; Lee et al., 1989; Perrella et al., 1992; Edwards et al., 1988; Mukoyama et al., 1991). Genetic disruption of the NPR-A receptor resulted in a renal unresponsiveness to ANP, impaired natriuretic response to acute volume expansion and sustained arterial hypertension (Lopez et al., 1995). Genetic disruption of ANP synthesis resulted in salt-sensitive hypertension (Takahashi et al., 1992). Moreover, overexpression models of ANP and BNP resulted in sustained hypotension, maintenance of sodium balance despite reductions in renal perfusion pressure and decreases in myocardial weight (DeBold et al., 1996; Stevens et al., 1996). These pharmacological and genetic manipulations of the endogenous NPS unequivocally support an important physiological role for the NPS in the humoral integration of the heart, kidney and vasculature in the control of sodium balance and arterial pressure.

Selective neurohumoral activation is a hallmark of early asymptomatic left ventricular dysfunction (ALVD). ALVD progresses to congestive heart failure (CHF). ANP and BNP are released from the atria and ventricles in response to atrial and ventricular stretch and serve to maintain a state of compensation despite myocardial dysfunction. The physiological mechanism of this compensation is activation of the NPR-A by ANP and BNP, resulting in the generation of the second messenger cGMP. NPR-A activation results in diverse biological responses in multiple organs, e.g., natriuresis, renin and aldosterone inhibition, and vasodilatation and lusitropic actions upon the cardiac myocyte.

Overt CHF is a state of avid sodium retention, enhanced vasoconstrictor and sodium-retaining activity and renal hyporesponsiveness to exogenous ANP. Nonetheless, acute natriuretic peptide receptor antagonism with HS-142-1 augments the magnitude of retention of sodium supporting a continuing natriuretic action of the endogenous NPS in overt CHF. Moreover, in a model of acute heart failure and in experimental ALVD employing active HS-142-1, decreased plasma and urinary cGMP, increased plasma renin activity and reduced sodium excretion to this NPR antagonism were observed (Stevens et al., 1996; Stevens et al., 1995; Stevens et al., 1994). In addition, in experimental ALVD in which the endogenous NPS was suppressed by removal of both atrial appendages, premature sodium retention, activation of both renin and aldosterone and an impaired natriuretic response to acute volume expansion were reported (Stevens et al., 1995).

Administration of BNP in animal models and humans with symptomatic heart failure has resulted in vasodilatory and natriuretic responses in the absence of deleterious neurohumoral activation (Marcus et al., 1996; Grantham et al., 1997). Thus, when taken together, the NPS emerges as a compensatory endocrine response to LV dysfunction to preserve a state of sodium balance without activation of the renin-angiotensin-aldosterone system and a limitation to the increase in cardiac filling pressures produced by impaired myocardial function. However, thc convenience and cost of systemic peptide delivery preclude BNP's easy long-term use as a therapy for LV dysfunction despite its potential to attenuate the progression of LV dysfunction based upon its unique and diverse properties.

Thus, what is needed is an improved method to inhibit or prevent cardiovascular disease, e.g., ALVD or heart failure.

### SUMMARY OF THE INVENTION

The invention provides a method to prevent or treat cardiovascular diseases such as atherosclerosis and its major complications: heart attack, heart failure and stroke, restenosis following angioplasty, hypertension, pulmonary hypertension and the vascular and cardiac adaptations to heart failure. Thus, the invention provides a method comprising administering to a mammal at risk of, or having, a cardiovascular disease an amount of a composition comprising a nucleic acid molecule, e.g., a DNA molecule which encodes BNP, DNP, or chimeras of ANP, CNP, BNP or DNP, effective to inhibit or prevent a cardiovascular disease, e.g., congestive heart failure. Both local, e.g., cardiac, and systemic, e.g., skeletal muscle, administration is envisioned. Skeletal myoblasts may be transduced ex vivo or in vivo. Ex vivo transduced myoblasts are then introduced to target animals via intramuscular (IM) injection. Direct injection of skeletal muscle has several advantages including the ease and safety of intramuscular injection and its ability to express transgenes from plasmid DNA. Local delivery may be accomplished by the intracoronary administration of a delivery vehicle such as recombinant adenovirus which encodes a natriuretic peptide, e.g., via a catheter, or intramyocardial delivery, e.g., during open heart surgery. Preferred mammals include, but are not limited to, canines, felines, ovines, bovines, swine, equines and primates, e.g., humans.

Thus, the invention provides a method to inhibit or prevent heart failure in a mammal. The method comprises administering to the mammal an effective amount of a composition comprising a nucleic acid molecule comprising a nucleic acid segment encoding brain natriuretic peptide or a chimera thereof in a delivery vehicle. In another embodiment of the invention, the mammal is administered an effective amount of a composition comprising a nucleic acid molecule comprising a nucleic acid segment encoding D-type natriuretic peptide or a chimera thereof in a delivery vehicle.

Preferably, the method inhibits or prevents the progression of ALVD to congestive heart failure. It is preferred that the nucleic acid molecule of the invention encodes a peptide having an activity similar to or greater than that of native BNP, i.e., the peptide is a potent natriuretic, diuretic, vasoactive and/or lusitropic hormone. It is also preferred that the therapeutic index of the encoded peptide is similar to, or greater than that of, native BNP. The degradation of BNP which is expressed in the mammal from the nucleic acid molecule of the invention may be inhibited by the administration of inhibitors of neutral endopeptidase (NEP), or the clearance of BNP from a mammal expressing a nucleic acid molecule of the invention may be accomplished with inhibitors of the clearance receptor, which may enhance local or circulating levels of BNP. The composition of the invention may include, for example, a plasmid comprising the nucleic acid molecule of the invention, or may include recombinant virus, e.g., recombinant adeno-associated viruses, adenoviruses or lentiviruses, which comprises the nucleic acid molecule of the invention, e.g., inserted into an adeno-associated virus vector, an adenovirus vector, or a lentivirus vector.

The invention also provides for expression cassettes encoding a natriuretic peptide or a chimera thereof, e.g., encoding portions of BNP and DNP. Such cassettes may also include viral sequences, e.g., sequences from an adenovirus, adeno-associated virus or a lentivirus.

Therefore, viral vectors are also provided by the invention. A preferred vector is an adenovirus vector comprising a nucleic acid molecule comprising a nicleic acid segment encoding D-type natriuretic peptide or a chimera thereof operably linked to transcriptional regulatory elements. Hence, the invention further provides a recombinant adenovirus comprising a DNA molecule comprising a DNA segment encoding a brain natriuretic peptide or a chimera thereof. Also provided is a recombinant adenovirus comprising a DNA molecule comprising a DNA segment encoding a D-type natriuretic peptide or a chimera thereof.

Also provided is an adeno-associated virus vector comprising a nucleic acid molecule comprising a nucleic acid segment encoding brain natriuretic peptide operably linked to transcriptional regulatory elements, and an adeno-associated virus vector comprising a nucleic acid molecule comprising a nucleic acid segment encoding D-type natriuretic peptide or a chimera thereof operably linked to transcriptional regulatory elements. Further provided are compositions and kits comprising the nucleic acid molecule(s), vector(s) or virus(es) of the invention.

The invention further provides a method to relax cardiac muscle. The method comprises administering to the mammal an effective amount of a composition comprising a nucleic acid molecule comprising a nucleic acid segment encoding brain natriuretic peptide or a chimera thereof in a delivery vehicle. In yet another embodiment of the invention, a mammal is administered an effective amount of a composition comprising a nucleic acid molecule comprising a nucleic acid segment encoding D-type natriuretic peptide or a chimera thereof in a delivery vehicle.

Further provided is a method to inhibit or prevent vasospasm. The method comprises administering to the mammal an effective amount of a composition comprising a nucleic acid molecule comprising a nucleic acid segment encoding brain natriuretic peptide or a chimera thereof in a delivery vehicle. In another embodiment, an effective amount of a composition comprising a nucleic acid molecule comprising a nucleic acid segment encoding D-type natriuretic peptide or a chimera thereof in a delivery vehicle is administered to the mammal.

In yet another embodiment of the invention, a composition comprising a nucleic acid molecule of the invention is employed in a method to inhibit or prevent atherosclerosis or in a method to inhibit or prevent vascular restenosis following percutaneous coronary intervention.

Further provided is a method to increase natriuretic peptide levels in a mammal. The method comprises administering to the mammal a composition comprising a nucleic acid molecule comprising a nucleic acid segment encoding a natriuretic peptide, e.g., BNP. Also provided is a method in which the mammal is administered an effective amount of a composition comprising a nucleic acid molecule comprising a nucleic acid segment encoding D-type natriuretic peptide or a chimera thereof in a delivery vehicle.

The invention also provides a method to detect progression of heart failure in a mammal subjected to brain natriuretic gene therapy. The method comprises monitoring brain natriuretic peptide levels in a mammal subjected to the administration of a composition comprising a nucleic acid molecule comprising a nucleic acid segment encoding brain natriuretic peptide. In a further embodiment of the invention, natriuretic peptide levels are monitored in a mammal subjected to the administration of a composition comprising a nucleic acid molecule comprising a nucleic acid segment encoding D-type natriuretic peptide or a chimera thereof.

Further provided is a method to inhibit or prevent pulmonary hypertension in mammal, in which the mammal is administered an effective amount of a composition comprising a nucleic acid molecule comprising a nucleic acid segment encoding natriuretic peptide or a chimera thereof in a delivery vehicle.

### BRIEF DESCRIPTION OF THE FIGURES

FIG. 1. DNA, mRNA and protein structure and processing of human BNP.

FIG. 2. Human (SEQ ID NO:9 encoded by SEQ ID NO:1) and canine (SEQ ID NO:3 encoded by SEQ ID NO:4) BNP. Conserved sequences are shaded.

FIG. 3. Plasmid map of pCMVint-hBNP. This plasmid expresses human BNP (HBNP) from cDNA driven by the CMV IE promoter/enhancer and the 5′untranslated sequence and first intron from CMV IE with a bovine growth hormone (BGH) polyadenylation site.

FIG. 4. Expression of HBNP from human embryonic kidney (293) cells. 293 cells at 50-60% confluence in 150 mm² dishes were transfected in triplicate with 20 μg of pCMVint-hBNP or pCMVint in combination with 40 μg of lipofectamine in Opti-MEM. Twenty-four hours later, the media was removed and the cells were washed and the media was replaced with 6 mls of serum-free DMEM for 48 hours at 37° C. Following this incubation, conditioned media was removed, spun at 12,000 rpm for 10 minutes to remove cellular contaminants and frozen in aliquots at −80° C. for later analysis by a specific radioimmunoassay for hBNP. *=p<0.01.

FIG. 5. Immunoblot analysis of hBNP from 293 cells. Conditioned media from the transfections with pCMVint-hBNP (lane 1) and pCMVint (lane 2) were analyzed by immunoblotting. Recombinant mature human BNP (Phoenix Pharmaceuticals, Mountain View, Calif.) was used as a control (lane 3). Molecular weights are indicated on the right.

FIG. 6. Stimulation of cGMP from canine glomerular cells by conditioned media from 293 cells transfected with pCMVint or pCMVint-hBNP. Canine glomerular cells were stimulated with conditioned media from transfections with pCMVint or pCMVint-hBNP in the presence of the phosphodiesterase inhibitor, IBMX (Sigma, St. Louis, Mo.). cGMP was analyzed by radioimmunoassay. *p<0.01.

FIG. 7. Plasmid map of pCMVint-gcBNP. This plasmid expresses canine BNP (cBNP) from genomic DNA driven by the CMV IE promoter/enhancer and the 5′ untranslated sequence and first intron from CMV IE with a bovine growth hormone (BGH) polyadenylation site.

FIG. 8. Expression of cBNP from human embryonic kidney (293) cells. 293 cells at 50-60% confluence in 150 mm² dishes were transfected in triplicate with 20 μg of pCMVint-gcBNP or pCMVint in combination with 40 μg of lipofectamine in Opti-MEM. Twenty-four hours later, the media was removed and the cells were washed and the media was replaced with 6 mls of serum-free DMEM for 48 hours at 37° C. Following this incubation, conditioned media was removed, spun at 12,000 rpm for 10 minutes to remove cellular contaminants and frozen in aliquots at −80° C. for later analysis by a specific radioimmunoassay for cBNP. *=p<0.01.

FIG. 9. Plasmid map of pAdCMVint-hBNP. This adenoviral shuttle plasmid containing Ad5 sequences (0-1 and 9.2-16.1) expresses human BNP (hBNP) from cDNA driven by the CMV IE promoter/enhancer and the 5′ untranslated sequence and first intron from CMV IE with a bovine growth hormone (BGH) polyadenylation site.

FIG. 10. Cloning strategy for development of plasmid expressing mature form of cBNP. To develop a plasmid that expresses mature cBNP directly, long oligonucleotides are synthesized, annealed, and filled in with DNA polymerase. The product is isolated, purified and cloned into pCMVint using native restriction sites.

FIG. 11. Exemplary codons.

FIG. 12. Preferred amino acid substitutions.

FIG. 13. Comparison of the amino acid sequence and structure of ANP (SEQ ID NO: 16), BNP (SEQ ID NO:9), CNP (SEQ ID NO: 17) and DNP (SEQ ID NO: 18).

FIG. 14. A) Amino acid sequence of human BNP (SEQ ID NO:2). B) DNA sequence encoding human BNP (SEQ ID NO:1).

### DETAILED DESCRIPTION OF THE INVENTION

Definitions

As used herein, the terms “isolated and/or purified” refer to in vitro preparation, isolation and/or purification of a therapeutic agent of the invention, so that it is not associated with in vivo substances. Thus, with respect to an “isolated nucleic acid molecule”, which includes a polynuclcotide of genomic, cDNA. or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. An isolated nucleic acid molecule means a polymeric form of nucleotides of at least 10 bases in length, either ribonucleotides or deoxynucleotides or a modified form of either type of nucleotide. The term includes single and double stranded forms of DNA. The term “oligonucleotide” referred to herein includes naturally occurring, and modified nucleotides linked together by naturally occurring, and non-naturally occurring oligonucleotide linkages. Oligonucleotides are a polynucleotide subset with 200 bases or fewer in length. Preferably, oligonucleotides are 10 to 60 bases in length and most preferably 12, 13, 14, 15, 16, 17, 18, 19, or 20 to 40 bases in length. Oligonucleotides are usually single stranded, e.g., for probes; although oligonucleotides may be double stranded, e.g., for use in the construction of a variant. Oligonucleotides of the invention can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phophoroselenoate, phosphorodiselenoate, phosphoroanilothioate, phosphoraniladate, phosphoroamidate, and the like. An oligonucleotide can include a label for detection, if desired.

The term “isolated polypeptide” means a polypeptide encoded by DNA or RNA, including synthetic DNA or RNA, or some combination thereof, which isolated polypeptide (1) is not associated with proteins found in nature, (2) is free of other proteins from the same source, e.g., free of human proteins, (3) is expressed by a cell from a different species, or (4) does not occur in nature.

The term “sequence homology” means the proportion of base matches between two nucleic acid sequences or the proportion amino acid matches between two amino acid sequences. When sequence homology is expressed as a percentage, e.g., 50%, the percentage denotes the proportion of matches over the length of sequence that is compared to some other sequence. Gaps (in either of; the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and more preferably not less than 19 matches out of 20 possible base pair matches (95%).

The term “selectively hybridize” means to detectably and specifically bind. Polynucleotides, oligonucleotides and fragments of the invention selectively hybridize to nucleic acid strands under hybridization and wash conditions that minimize appreciable amounts of detectable binding to nonspecific nucleic acids. High stringency conditions can be used to achieve selective hybridization conditions as known in the art and discussed herein. Generally, the nucleic acid sequence homology between the polynucleotides, oligonucleotides, and fragments of the invention and a nucleic acid sequence of interest is at least 65%, and more typically with preferably increasing homologies of at least about 70%, about 90%, about 95%, about 98%, and 100%.

Two amino acid sequences are homologous if there is a partial or complete identity between their sequences. For example, 85% homology means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) are homologous, as this term is used herein, if they have an alignment score of at more than 5 (in standard deviation units) using the program ALIGN with the mutation data matrix and a gap penalty of 6 or greater. See Dayhoff, M. O., in Atlas of Protein Sequence and Structure, 1972, volume 5, National Biomedical Research Foundation, pp. 101-110, and Supplement 2 to this volume, pp. 1-10. The two sequences or parts thereof are more preferably homologous if their amino acids are greater than or equal to 50% identical when optimally aligned using the ALIGN program.

The term “corresponds to” is used herein to mean that a polynucleotide sequence is homologous (i.e., is identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “TATAC” corresponds to a reference sequence “TATAC” and is complementary to a reference sequence “GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48; 443, by the search for similarity method of Pearson and Lipman (1988) Proc. Natl. Acad. Sci. (U.S.A.) 85; 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denote a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 20-50 nucleotides, wherein the percentage of sequence identity is calculated by comparing the reference sequence to the polynucleotide sequence which may include deletions or additions which total 20 percent or less of the reference sequence over the window of comparison.

As applied to polypeptides, the term “substantial identity” means that two peptide sequences, when optimally aligned, such as by the programs GAP or BESTFIT using default gap weights, share at least about 80 percent sequence identity, preferably at least about 90 percent sequence identity, more preferably at least about 95 percent sequence identity, and most preferably at least about 99 percent sequence identity.

I. BNP Gene Organization and Protein Processing

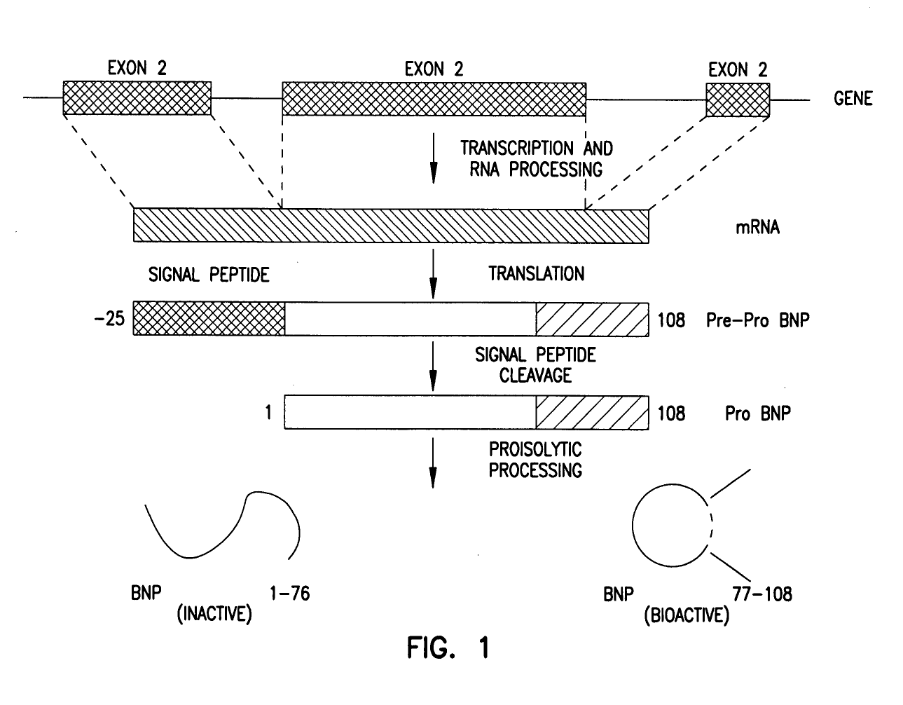

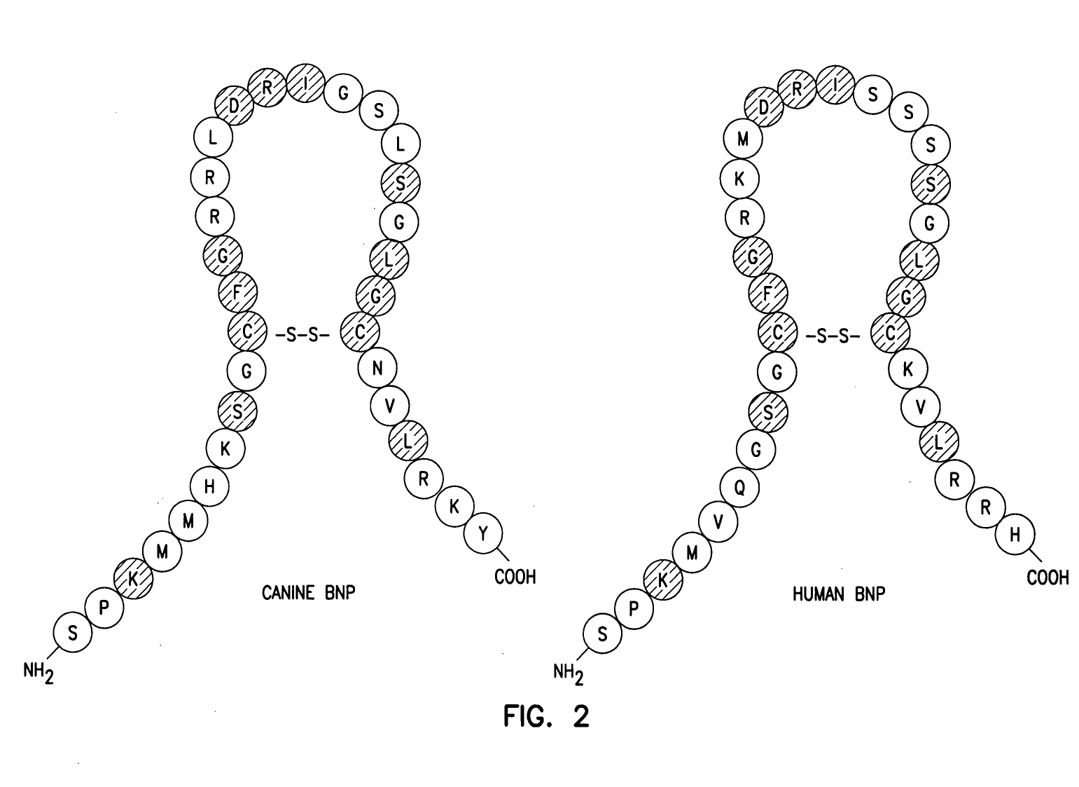

The natriuretic peptides are a similar but genetically distinct family of proteins. The gene for human BNP contains 3 exons (FIG. 1) and is found on chromosome 1. The first exon encodes for a signal peptide (26 amino acids). The immature form, pro-BNP, contains 108 amino acids while the mature form of BNP contains 32 amino acids. The gene for the canine form has similar structure and the protein requires similar processing (FIG. 2). Like ANP, BNP has a highly conserved disulfide bridge in the middle of the molecule with short stretches of amino acids extending from either terminal (Gardner, 1994). Unlike ANP which is processed when it is secreted from granules in atrial myocytes, the steps involved in the processing of BNP remain unclear. In cardiac atria and ventricles, high and low molecular weight forms of BNP coexist. In human plasma, pro-BNP is the predominant molecular form (DeBold et al., 1981). Even in patients with CHF and following myocardial infarction with high levels of immunoreactive BNP, pro-BNP remains a predominant species (Tateyama et al., 1992). The biologic activity of pro-BNP as compared to mature BNP remains uncertain although it is thought to be active (Tateyama et al., 1992).

The processing of human BNP involves peptide cleavage downstream of a consensus sequence of -Arg-X-X-Arg- (RXXR; SEQ ID NO:5) (FIG. 1). This cleavage site is also found in many other vasoactive peptides including CNP, big endothelia and adrenomedullin (Sawada et al., 1997a). This motif is cleaved by members of the Kex2 family endoproteases of which furin is a member. Furin is localized on the trans-Golgi networks of most cells (Sawada et al., 1997a). In rat hearts following myocardial infarction, the expression of furin parallels the biphasic expression of BNP (Sawada et al., 1997a). Stretch of rat cardiomyocytes induces expression of BNP and furin and inhibition of furin blocks the processing of BNP (Sawada et al., 1997b). Thus, furin may process the pro form of BNP. The relative activity and expression of furin and its role in expression of NP in cardiac and noncardiac tissue in normal and diseased states has not been fully determined. Furthermore, processing may differ between atrial and ventricular sites of BNP production. Ventricular myocytes do not contain secretory granules and may secrete pro-BNP through a constitutive pathway (Sawada et al., 1997a). Thus, co-expression or co-regulation of putative processing enzymes may be important in strategies to overexpress active forms of BNP from cardiac and noncardiac cells.

II. Nucleic Acid Molecules of the Invention

A. Sources of the Nucleic Acid Molecules of the Invention

Sources of nucleotide sequences from which the present nucleic acid molecules encoding a NP, e.g., BNP or DNP, or a variant thereof, or the nucleic acid complement thereof, include total or polyA⁺RNA from any eukaryotic, preferably mammalian, e.g., human, rat, mouse, canine, bovine, equine, ovine, caprine, feline, more preferably primate, e.g., human, cellular source from which cDNAs can be derived by methods known in the art. Other sources of the DNA molecules of the invention include genomic libraries derived from any eukaryotic, preferably mammalian, cellular source, e.g., those exemplified above. Moreover, the present DNA molecules may be prepared in vitro, or by subcloning a portion of a DNA segment that encodes a particular NP.

B. Isolation of a Gene Encoding a NP

A nucleic acid molecule encoding a NP can be identified and isolated using standard methods, as described by Sambrook et al., Molecular Cloning: A Laboratory Manual, Cold Spring Harbor, N.Y. (1989). For example, reverse-transciptase PCR (RT-PCR) can be employed to isolate and clone BNP or DNP cDNAs. Oligo-dT can be employed as a primer in a reverse transcnptase reaction to prepare first-strand cDNAs from isolated RNA which contains RNA sequences of interest, e.g., total RNA isolated from human tissue. RNA can be isolated by methods known to the art, e.g., using TRIZOL™ reagent (GIBCO-BRL/Life Technologies, Gaithersburg, Md.). Resultant first-strand cDNAs are then amplified in PCR reactions.

“Polymerase chain reaction” or “PCR” refers to a procedure or technique in which amounts of a preselected fragment of nucleic acid, RNA and/or DNA, are amplified as described in U.S. Pat. No. 4,683,195. Generally, sequence information from the ends of the region of interest or beyond is employed to design oligonucleotide primers comprising at least 7-8 nucleotides. These primers will be identical or similar in sequence to opposite strands of the template to be amplified. PCR can be used to amplify specific RNA sequences, specific DNA sequences from total genomic DNA, and cDNA transcribed from total cellular RNA, bacteriophage or plasmid sequences, and the like. See generally Mullis et al., Cold Spring Harbor Symp. Quant. Biol., 51, 263 (1987); Erlich, ed., PCR Technology, (Stockton Press, N.Y., 1989). Thus, PCR-based cloning approaches rely upon conserved sequences deduced from alignments of related gene or polypeptide sequences.

Primers are made to correspond to highly conserved regions of polypeptides or nucleotide sequences which were identified and compared to generate the primers, e.g., by a sequence comparison of other eukaryotic BNP or DNPs. One primer is prepared which is predicted to anneal to the antisense strand, and another primer prepared which is predicted to anneal to the sense strand, of a DNA molecule which encodes, for example, a BNP or DNP.

The products of each PCR reaction are separated via an agarose gel and all consistently amplified products are gel-purified and cloned directly into a suitable vector, such as a known plasmid vector. The resultant plasmids are subjected to restriction endonuclease and dideoxy sequencing of double-stranded plasmid DNAs.

Another approach to identify, isolate and clone cDNAs which encode a NP is to screen a cDNA library. Screening for DNA fragments that encode all or a portion of a cDNA encoding a NP can be accomplished by probing the library with a probe which has sequences that are highly conserved between genes believed to be related to the NP, e.g., the homolog of a particular NP from a different species, or by screening of plaques for binding to antibodies that specifically recognize BNP or DNP. DNA fragments that bind to a probe having sequences which are related to NP, or which are immunoreactive with antibodies to NP, can be subcloned into a suitable vector and sequenced and/or used as probes to identify other cDNAs encoding all or a portion of the NP, e.g., BNP or DNP.

As used herein, the terms “isolated and/or purified” refer to in vitro isolation of a DNA or polypeptide molecule from its natural cellular environment, and from association with other components of the cell, such as nucleic acid or polypeptide, so that it can be sequenced, replicated, and/or expressed. For example, “isolated BNP nucleic acid” is RNA or DNA containing greater than 9, preferably 36, and more preferably 45 or more, sequential nucleotide bases that encode at least a portion of BNP, or a variant thereof, or a RNA or DNA complementary thereto, that is complementary or hybridizes, respectively, to RNA or DNA encoding BNP and remains stably bound under stringent conditions, as defined by methods well known in the art, e.g., in Sambrook et al., supra. Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell.

As used herein, the term “recombinant nucleic acid” or “preselected nucleic acid,” e.g., “recombinant DNA sequence or segment” or “preselected DNA sequence or segment” refers to a nucleic acid, e.g., to DNA, that has been derived or isolated from any appropriate tissue source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonuclcascs, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. See Lawn et al., Nucleic Acids Res., 9, 6103 (1981), and Goeddel et al., Nucleic Acids Res., 8, 4057 (1980). Therefore, “preselected DNA” includes completely synthetic DNA sequences, semi-synthetic DNA sequences, DNA sequences isolated from biological sources, and DNA sequences derived from RNA, as well as mixtures thereof.

As used herein, the term “derived” with respect to a RNA molecule means that the RNA molecule has complementary sequence identity to a particular DNA molecule.

C. Variants of the Nucleic Acid Molecules of the Invention

Nucleic acid molecules encoding amino acid sequence variants of NP are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the NP.

Oligonucleotide-mediated mutagenesis is a preferred method for preparing amino acid substitution variants of NP. This technique is well known in the art as described by Adelman et al., DNA, 2, 183 (1983). Briefly, for example, BNP DNA is altered by hybridizing an oligonucleotide encoding the desired mutation to a DNA template, where the template is the single-stranded form of a plasmid or bacteriophage containing the unaltered or native DNA sequence of the BNP. After hybridization, a DNA polymerase is used to synthesize an entire second complementary strand of the template that will thus incorporate the oligonucleotide primer, and will code for the selected alteration in the BNP DNA.

Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al., Proc. Natl. Acad. Sci., U.S.A., 75, 5765 (1978).

The DNA template can be generated by those vectors that are either derived from bacteriophage M13 vectors (the commercially available M13mp18 and M13mp19 vectors are suitable), or those vectors that contain a single-stranded phage origin of replication as described by Viera et al., Meth. Enzymol., 153, 3 (1987). Thus, the DNA that is to be mutated may be inserted into one of these vectors to generate single-stranded template. Production of the single-stranded template is described in Sections 4.21-4.41 of Sambrook et al., Molecular Cloning: A Laboratory Manual (Cold Spring Harbor Laboratory Press, N.Y. 1989).

Alternatively, single-stranded DNA template may be generated by denaturing double-stranded plasmid (or other) DNA using standard techniques.

For alteration of the native DNA sequence (to generate amino acid sequence variants, for example), the oligonucleotide is hybridized to the single-stranded template under suitable hybridization conditions. A DNA polymerizing enzyme, usually the Klenow fragment of DNA polymerase I, is then added to synthesize the complementary strand of the template using the oligonucleotide as a primer for synthesis. A heteroduplex molecule is thus formed such that one strand of DNA encodes the mutated form of, for example, BNP, and the other strand (the original template) encodes the native, unaltered sequence of BNP. This heteroduplex molecule is then transformed into a suitable host cell, usually a prokaryote such as E. coli JM101. After the cells are grown, they are plated onto agarose plates and screened using the oligonucleotide primer radiolabeled with 32-phosphate to identify the bacterial colonies that contain the mutated DNA. The mutated region is then removed and placed in an appropriate vector for peptide or polypeptide production, generally an expression vector of the type typically employed for transformation of an appropriate host.

The method described immediately above may be modified such that a homoduplex molecule is created wherein both strands of the plasmid contain the mutations(s). The modifications are as follows: The single-stranded oligonucleotide is annealed to the single-stranded template as described above. A mixture of three deoxyribonucleotides, deoxyriboadenosine (dATP), deoxyriboguanosine (dGTP), and deoxyribothymidine (dTTP), is combined with a modified thiodeoxyribocytosine called dCTP-(αS) (which can be obtained from the Amersham Corporation). This mixture is added to the template-oligonucleotide complex. Upon addition of DNA polymerase to this mixture, a strand of DNA identical to the template except for the mutated bases is generated. In addition, this new strand of DNA will contain dCTP-(αS) instead of dCTP, which serves to protect it from restriction endonuclease digestion.

After the template strand of the double-stranded heteroduplex is nicked with an appropriate restriction enzyme, the template strand can be digested with ExoIII nuclease or another appropriate nuclease past the region that contains the site(s) to be mutagenized. The reaction is then stopped to leave a molecule that is only partially single-stranded. A complete double-stranded DNA homoduplex is then formed using DNA polymerase in the presence of all four deoxyribonucleotide triphosphates, ATP, and DNA ligase. This homoduplex molecule can then be transformed into a suitable host cell such as E. coli JM101.

For example, a preferred embodiment of the invention is an isolated and purified DNA molecule comprising a preselected DNA segment encoding human BNP comprising SEQ ID NO:2 (prepro form), SEQ ID NO:7 (pro form), or SEQ ID NO:9 (mature form), wherein the DNA segment comprises SEQ ID NO: 1, SEQ ID NO:6, or SEQ ID NO:8, respectively, or variants of SEQ ID NO: 1, SEQ ID NO:6 or SEQ ID NO:8 having nucleotide substitutions which are “silent” (see FIG. 11). That is, when silent nucleotide substitutions are present in a codon, the same amino acid is encoded by the codon with the nucleotide substitution as is encoded by the codon without the substitution. For example, valine is encoded by the codon GTT, GTC, GTA and GTG. A variant of SEQ ID NO:1 at the fifth to the last codon (GTG in SEQ ID NO:1) includes the substitution of GTT, GTA or GTC for GTG. Other “silent” nucleotide substitutions in which can encode SEQ ID NO:1 can be ascertained by reference to FIG. 11 and page D1 in Appendix D in Sambrook et al., Molecular Cloning A Laboratory Manual (1989). Nucleotide substitutions can be introduced into DNA segments by methods well known to the art. See, for example, Sambrook et al., supra. Likewise, nucleic acid molecules encoding other mammalian, preferably human, NPs may be modified in a similar manner. Nucleic acid molecules falling within the scope of the invention include those which hybridize under stringent hybridization conditions to SEQ ID NO: 1, SEQ ID NO:6, or SEQ ID NO:8. Moderate and stringent hybridization conditions are well known to the art, see, for example, sections 9.47-9.51 of Sambrook et al. (Molecular Cloning: A Laboratory Manual, Cold Spring Harbor Laboratory, Cold Spring Harbor, N.Y. (1989)). For example, stringent conditions are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as fornamide during hybridization e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium phosphate,5 ×Denhardt's solution, sonicated salmon sperm DNA (50 μ/ml), 0.1% sodium dodecylsulfate (SDS), and 10% dextran sulfate at 42° C., with washes at 42° C. in 0.2 ×SSC and 0.1% SDS.

Thus, it is also envisioned that one or more of the residues of the peptide encoded by the nucleic acid molecules of the invention can be altered, so long as the peptide variant is biologically active. It is preferred that the variant has at least about 10% of the biological activity of the corresponding non-variant peptide, e.g., a peptide having SEQ ID NO:2, SEQ ID NO:7 or SEQ ID NO:9. Conservative amino acid substitutions are preferred—that is, for example, aspartic-glutamic as acidic amino acids; lysine/arginine/histidine as basic amino acids; leucine/isoleucine, methionine/valine, alanine/valine as hydrophobic amino acids; serine/glycine/alanine/threonine as hydrophilic amino acids. Conservative amino acid substitution also includes groupings based on side chains. For example, a group of amino acids having aliphatic side chains is glycine, alanine, valine, leucine, and isoleucine; a group of amino acids having aliplhatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional peptide can readily be determined by assaying the specific activity of the peptide variant. Assays are described in detail herein.

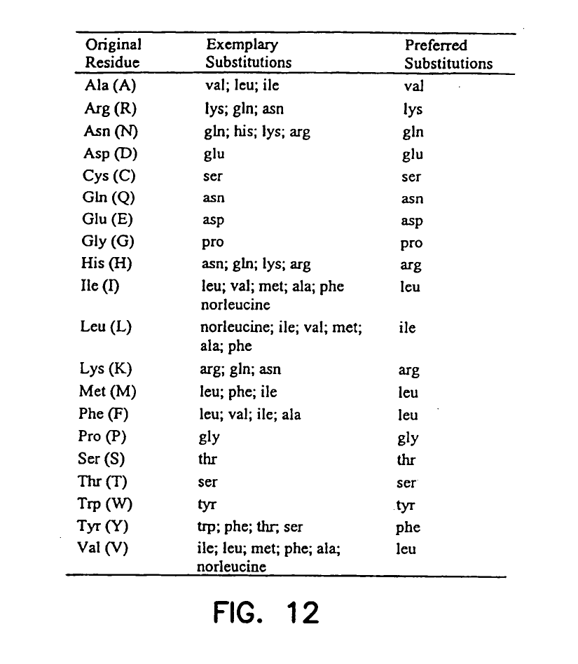

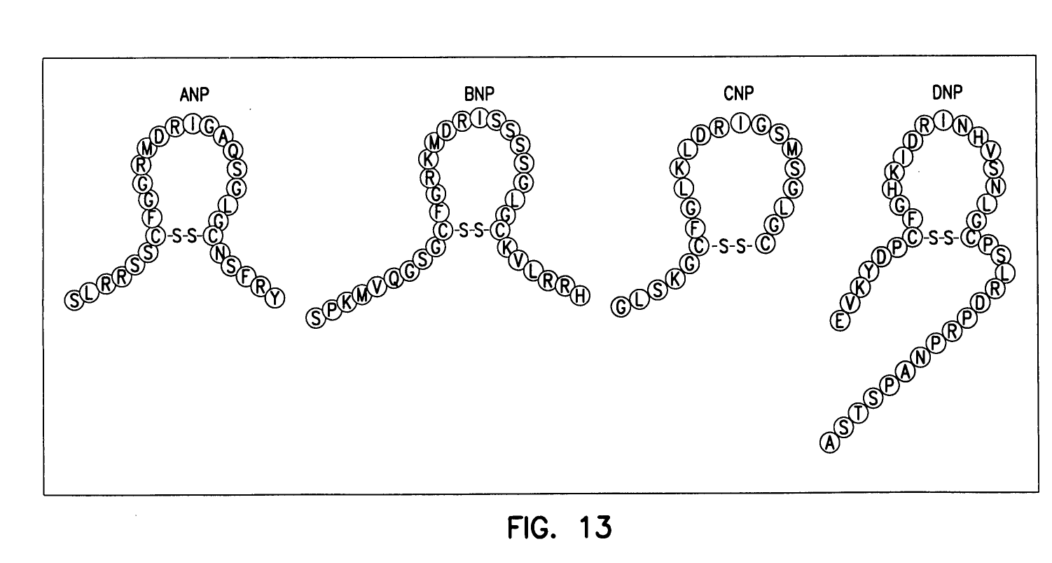

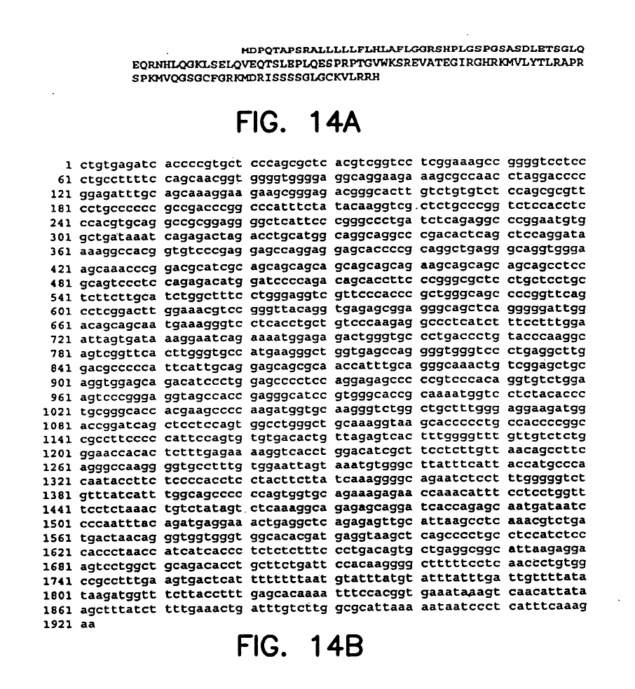

Conservative substitutions are shown in FIG. 12 under the heading of exemplary substitutions. More preferred substitutions are under the heading of preferred substitutions. After the substitutions are introduced, the variants are screened for biological activity.

Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic; trp, tyr, phe.

The invention also envisions peptide variants with non-conservative substitutions. Non-conservative substitutions entail exchanging a member of one of the classes described above for another.

III. Preparation of Agents Falling Within the Scope of the Invention

A. Chimeric Fxpression Cassettes

To prepare expression cassettes for transformation herein, the recombinant or preselected DNA sequence or segment may be circular or linear, double-stranded or single-stranded. A preselected DNA sequence which encodes an RNA sequence that is substantially complementary to a mRNA sequence encoding a NP, such as BNP or DNP, is typically a “sense” DNA sequence cloned into a cassette in the opposite orientation (i.e., 3′ to 5′ rather than 5′ to 3′). Generally, the preselected DNA sequence or segment is in the form of chimeric DNA, such as plasmid DNA, that can also contain coding regions flanked by control sequences which promote the expression of the preselected DNA present in the resultant cell line.

As used herein, “chimeric” means that a vector comprises DNA from at least two different species, or comprises DNA from the same species, which is linked or associated in a manner which does not occur in the “native” or wild type of the species.

Aside from preselected DNA sequences that serve as transcription units for NP, e.g., BNP or DNP, or portions thereof, a portion of the preselected DNA may be untranscribed, serving a regulatory or a structural function. For example, the preselected DNA may itself comprise a promoter that is active in mammalian cells, or may utilize a promoter already present in the genome that is the transformation target. Such promoters include the CMV promoter, as well as the

SV40 late promoter and retroviral LTRs (long terminal repeat elements), although many other promoter elements well known to the art may be employed in the practice of the invention.

Other elements functional in the host cells, such as introns, enhancers, polyadenylation sequences and the like, may also be a part of the preselected DNA. Such elements may or may not be necessary for the function of the DNA, but may provide improved expression of the DNA by affecting transcription, stability of the mRNA, or the like. Such elements may be included in the DNA as desired to obtain the optimal performance of the transforming DNA in the cell.

“Control sequences” is defined to mean DNA sequences necessary for the expression of an operably linked coding sequence in a particular host organism. The control sequences that are suitable for prokaryotic cells, for example, include a promoter, and optionally an operator sequence, and a ribosome binding site. Eukaryotic cells are known to utilize promoters, polyadenylation signals, and enhancers.

“Operably linked” is defined to mean that the nucleic acids are placed in a functional relationship with another nucleic acid sequence. For example, DNA for a presequence or secretory leader is operably linked to DNA for a peptide or polypeptide if it is expressed as a preprotein that participates in the secretion of the peptide or polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked arc contiguous and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers arc used in accord with conventional practice.

The preselected DNA to be introduced into the cells further will generally contain either a selectable marker gene or a reporter gene or both to facilitate identification and selection of transformed cells from the population of cells sought to be transformed. Alternatively, the selectable marker may be carried on a separate piece of DNA and used in a co-transformation procedure. Both selectable markers and reporter genes may be flanked with appropriate regulatory sequences to enable expression in the host cells. Useful selectable markers are well known in the art and include, for example, antibiotic and herbicide-resistance genes, such as neo, hpt, dhfr, bar, aroA, dapA and the like. See also, the genes listed on Table 1 of Lundquist et al. (U.S. Pat. No. 5,848,956).

Reporter genes are used for identifying potentially transformed cells and for evaluating the functionality of regulatory sequences. Reporter genes which encode for easily assayable proteins are well known in the art. In general, a reporter gene is a gene which is not present in or expressed by the recipient organism or tissue and which encodes a protein whose expression is manifested, by some easily detectable property, e.g., enzymatic activity. Preferred genes include the chloramphenicol acetyl transferase gene (cat) from Tn9 of E. coli, the beta-glucuronidase gene (gus) of the uidA locus of E. coli, and the luciferase gene from firefly Photinus pyralis. Expression of the reporter gene is assayed at a suitable time after the DNA has been introduced into the recipient cells.

The general methods for constructing recombinant DNA which can transform target cells are well known to those skilled in the art, and the same compositions and methods of construction may be utilized to produce the DNA useful herein. For example, J. Sambrook et al., Molecular Cloning: A Laboratory Manual, Cold Spring Harbor Laboratory Press (2nd ed., 1989), provides suitable methods of construction.

B. Transformation into Host Cells

The recombinant DNA can be readily introduced into the host cells, e.g., mammalian, bacterial, yeast or insect cells, by transfection with an expression vector comprising DNA encoding NP, a variant thereof or its complement, by any procedure useful for the introduction into a particular cell, e.g., physical or biological methods, to yield a transformed cell having the recombinant DNA stably integrated into its genome, so that the DNA molecules, sequences, or segments, of the present invention are expressed by the host cell.

Physical methods to introduce a preselected DNA into a host cell include calcium phosphate precipitation, lipofection, particle bombardment, microinjection, electroporation, and the like. Biological methods to introduce the DNA of interest into a host cell include the use of DNA and RNA viral vectors. The main advantage of physical methods is that they are not associated with pathological or oncogenic processes of viruses. However, they are less precise, often resulting in multiple copy insertions, random integration, disruption of foreign and endogenous gene sequences, and unpredictable expression. For mammalian gene therapy, viral vectors have become the most widely used method for introducing genes into mammalian, e.g., human, cells. Viral vectors can be derived from poxviruses, herpes simplex virus I, adenoviruses and adeno-associated viruses, retroviruses, lentiviruses and the like. A preferred embodiment of the invention is the use of adenoviral vectors to introduce a NP, e.g., BNP, DNP, or a chimeric NP, gene of the invention to a mammalian host.

As used herein, the temi “cell line” or “host cell” is intended to refer to well-characterized homogenous, biologically pure populations of cells. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources. Generally, the preselected DNA sequence is related to a DNA sequence which is resident in the genome of the host cell but is not expressed, or not highly expressed, or, alternatively, overexpressed.

“Transfected” or “transformed” is used herein to include any host cell or cell line, the genome of which has been altered or augmented by the presence of at least one preselected DNA sequence, which DNA is also referred to in the art of genetic engineering as “heterologous DNA,” “recombinant DNA,” “exogenous DNA,” “genetically engineered,” “non-native,” or “foreign DNA,” wherein said DNA was isolated and introduced into the genome of the host cell or cell line by the process of genetic engineering. The host cells of the present invention are typically produced by transfection with a DNA sequence in a plasmid expression vector, a viral expression vector, or as an isolated linear DNA sequence. Preferably, the transfected DNA is a chromosomally integrated recombinant DNA sequence, which comprises a gene encoding NP or its complement, which host cell may or may not express significant levels of autologous or “native” NP.

To confirm the presence of the preselected DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular BNP or DNP, e.g., by immunological means (immunoassays, such as ELISA and Western blot) or by assays described herein to identify agents falling within the scope of the invention.

To detect and quantitate RNA produced from introduced preselected DNA segments, RT-PCR may be employed. In this application of PCR, it is first necessary to reverse transcribe RNA into DNA, using enzymes such as reverse transcriptase, and then through the use of conventional PCR techniques amplify the DNA. In most instances PCR techniques, while useful, will not demonstrate integrity of the RNA product. Further information about the nature of the RNA product may be obtained by Northern blotting. This technique demonstrates the presence of an RNA species and gives information about the integrity of that RNA. The presence or absence of an RNA species can also be determined using dot or slot blot Northern hybridizations. These techniques are modifications of Northern blotting and only demonstrate the presence or absence of an RNA species.

While Southern blotting and PCR may be used to detect the preselected DNA segment in question, they do not provide information as to whether the preselected DNA segment is being expressed. Expression may be evaluated by specifically identifying the peptide products of the introduced preselected DNA sequences or evaluating the phenotypic changes brought about by the expression of the introduced preselected DNA segment in the host or host cell.

IV. Dosages Formulations and Routes of Administration of the Agents of the Invention

The therapeutic agents of the invention, are preferably administered at dosages of at least about 0.01 to about 100 mg/kg, more preferably about 0.1 to about 50 mg/kg, and even more preferably about 0.1 to about 30 mg/kg, of body weight, although other dosages may provide beneficial results. The amount administered will vary depending on various factors including, but not limited to, the nucleic acid molecule of the invention chosen, the disease, and whether prevention or treatment is to be achieved.

Administration of sense or antisense nucleic acid molecule may be accomplished through the introduction of cells transformed with an expression cassette comprising the nucleic acid molecule (see, for example, WO 93/02556) or the administration of the nucleic acid molecule (see, for example, Felgner et al., U.S. Pat. No. 5,580,859, Pardoll et al., Immunity, 3, 165 (1995); Stevenson et al., Immunol., Rev., 145, 211 (1995); Molling, J. Mol. Med., 75, 242 (1997); Donnelly et al., Ann. N.Y. Acad. Sci., 772, 40 (1995); Yang et al., Mol. Med. Today, 2, 476 (1996); Abdallah et al., Biol Cell, 85, 1 (1995); Wolff et al., Science, 247, 1465 (1990); Tripathy et al., PNAS, 91, 11557 (1994); Tripathy et al., PNAS, 93, 10876 (1996a); Tripathy et al., Nature Med., 2, 545 (1996b); Tsurumi et al., Cir., 94, 3281 (1996); Baumgartner et al., Circulation, 96, 1 (1997); Lin et al., Hypertension, 26, 847 (1990)). Pharmaceutical formulations, dosages and routes of administration for nucleic acids are generally disclosed, for example, in Feigner et al., supra.

Administration of recombinant adenovirus to deliver the expression cassette or nucleic acid molecule of the invention may be accomplished by any method known to the art. See, for example, Guzman et al., Circ. Res., 73, 1202 (1993); Kass-Eisler et al., PNAS, 90, 11498 (1993); and Giordano et al., Med, 2, 534 (1996).

The amount of therapeutic agent administered is selected to treat a particular indication. The therapeutic agents of the invention are also amenable to chronic use for prophylactic purposes. Both local and systemic administration are envisioned.

Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses.

One or more suitable unit dosage forms comprising the therapeutic agents of the invention, which, as discussed below, may optionally be formulated for sustained release, can be administered by a variety of routes including oral, or parenteral, including by rectal, buccal, vaginal and sublingual, transdermal, subcutaneous, intravenous, intramuscular, intraperitoneal, intrathoracic, intracoronary intrapulmonary and intranasal routes. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are preferably combined with a pharmaceutically acceptable carrier, diluent or excipient to form a pharmaceutical formulation, or unit dosage form. The total active ingredients in such formulations comprise from 0.1 to 99.9% by weight of the formulation. By “pharmaceutically acceptable” it is meant the carrier, diluent, excipient, and/or salt must be compatible with the other ingredients of the formulation, and not deleterious to the recipient thereof. The active ingredient for oral administration may be present as a powder or as granules; as a solution, a suspension or an emulsion; or in achievable base such as a synthetic resin for ingestion of the active ingredients from a chewing gum. The active ingredient may also be presented as a bolus, electuary or paste.

Formulations suitable for vaginal administration may be presented as pessaries, tampons, creams, gels, pastes, douches, lubricants, foams or sprays containing, in addition to the active ingredient, such carriers as are known in the art to be appropriate. Formulations suitable for rectal administration may be presented as suppositories.

Pharmaceutical formulations containing the therapeutic agents of the invention can be prepared by procedures known in the art using well known and readily available ingredients. For example, the agent can be formulated with common excipients, diluents, or carriers, and formed into tablets, capsules, suspensions, powders, and the like. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

For example, tablets or caplets containing the agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, and zinc stearate, and the like. Hard or soft gelatin capsules containing an agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric coated caplets or tablets of an agent of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder fonn, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to, prepare solutions using one or more organic solvent(s) that is/are acceptable from the physiological standpoint; chosen, in addition to water, from solvents such as acetone, ethanol, isopropyl alcohol, glycol ethers such as the products sold under the name “Dowanol”, polyglycols and polyethylene glycols, C₁-C₄ alkyl esters of short-chain acids, preferably ethyl or isopropyl lactate, fatty acid triglycerides such as the products marketed under the name “Miglyol”, isopropyl myristate, animal, mineral and vegetable oils and polysiloxanes.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

For example, among antioxidants, t-butylhydroquinone, butylated hydroxyanisole, butylated hydroxytoluene and a-tocopherol and its derivatives may be mentioned. The galenical forms chiefly conditioned for topical application take the form of creams, milks, gels, dispersion or microemulsions, lotions thickened to a greater or lesser extent, impregnated pads, ointments or sticks, or alternatively the form of aerosol formulations in spray or foam form or alternatively in the form of a cake of soap.

Additionally, the agents are well suited to formulation as sustained release dosagc forms and the like. The formulations can be so constituted that they release the active ingredient only or preferably in a particular part of the intestinal or respiratory tract, possibly over a period of time. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, and the like.

The therapeutic agents of the invention can be delivered via patches for transdcrmal administration. See U.S. Pat. No. 5,560,922 for examples of patches suitable for transdermal delivery of a therapeutic agent. Patches for transdermal delivery can comprise a backing layer and a polymer matrix which has dispersed or dissolved therein a therapeutic agent, along with one or more skin permeation enhancers. The backing layer can be made of any suitable material which is impermeable to the therapeutic agent. The backing layer serves as a protective cover for the matrix layer and provides also a support function. The backing can be formed so that it is essentially the same size layer as the polymer matrix or it can be of larger dimension so that it can extend beyond the side of the polymer matrix or overlay the side or sides of the polymer matrix and then can extend outwardly in a manner that the surface of the extension of the backing layer can be the base for an adhesive means. Alternatively, the polymer matrix can contain, or be formulated of, an adhesive polymer, such as polyacrylate or acrylate/vinyl acetate copolymer. For long-term applications it might be desirable to use microporous and/or breathable backing laminates, so hydration or maceration of the skin can be minimized.

Examples of materials suitable for making the backing layer are films of high and low density polyethylene, polypropylene, polyurethane, polyvinylchloride, polyesters such as poly(ethylene phthalate), metal foils, metal foil laminates of such suitable polymer films, and the like. Preferably, the materials used for the backing layer are laminates of such polymer films with a metal foil such as aluminum foil. In such laminates, a polymer film of the laminate will usually be in contact with the adhesive polymer matrix.

The backing layer can be any appropriate thickness which will provide the desired protective and support functions. A suitable thickness will be from about 10 to about 200 microns.

Generally, those polymers used to form the biologically acceptable adhesive polymer layer are those capable of forming shaped bodies, thin walls or coatings through which therapeutic agents can pass at a controlled rate. Suitable polymers are biologically and pharmaceutically compatible, nonallergenic and insoluble in and compatible with body fluids or tissues with which the device is contacted. The use of soluble polymers is to be avoided since dissolution or erosion of the matrix by skin moisture would affect the release rate of the therapeutic agents as well as the capability of the dosage unit to remain in place for convenience of removal.

Exemplary materials for fabricating the adhesive polymer layer include polyethylene, polypropylene, polyurethane, ethylene/propylene copolymers, ethylene/ethylacrylate copolymers, ethylene/vinyl acetate copolymers, silicone elastomers, especially the medical-grade polydimethylsiloxanes, neoprene rubber, polyisobutylene, polyacrylates, chlorinated polyethylene, polyvinyl chloride, vinyl chloride-vinyl acetate copolymer, crosslinked polymethacrylate polymers (hydrogel), polyvinylidene chloride, poly(ethylene terephtlhalate), butyl rubber, epichlorohydrin rubbers, ethylenvinyl alcohol copolymers, ethylene-vinyloxyethanol copolymers; silicone copolymers, for example, polysiloxane-polycarbonate copolymers, polysiloxanepolyethylene oxide copolymers, polysiloxane-polymethacrylate copolymers, polysiloxane-alkylene copolymers (e.g., polysiloxane-ethylene copolymers), polysiloxane-alkylenesilane copolymers (e.g., polysiloxane-ethylenesilane copolymers), and the like; cellulose polymers, for example methyl or ethyl cellulose, hydroxy propyl methyl cellulose, and cellulose esters; polycarbonates; polytetrafluoroethylene; and the like.

Preferably, a biologically acceptable adhesive polymer matrix should be selected from polymers with glass transition temperatures below room temperature. The polymer may, but need not necessarily, have a degree of crystallinity at room temperature. Cross-linking monomeric units or sites can be incorporated into such polymers. For example, cross-linking monomers can be incorporated into polyacrylate polymers, which provide sites for cross-linking the matrix after dispersing the therapeutic agent into the polymer. Known cross-linking monomers for polyacrylate polymers include polymethacrylic esters of polyols such as butylene diacrylate and dimethacrylate, trimethylol propane trimethacrylate and the like. Other monomers which provide such sites include allyl acrylate, allyl methacrylate, diallyl maleate and the like.

Preferably, a plasticizer and/or humectant is dispersed within the adhesive polymer matrix. Water-soluble polyols are generally suitable for this purpose. Incorporation of a humectant in the formulation allows the dosage unit to absorb moisture on the surface of skin which in turn helps to reduce skin irritation and to prevent the adhesive polymer layer of the delivery system from failing.

Therapeutic agents released from a transdermal delivery system must be capable of penetrating each layer of skin. In order to increase the rate of permeation of a therapeutic agent, a transdermal drug delivery system must be able in particular to increase the permeability of the outermost layer of skin, the stratum corneuni, which provides the most resistance to the penetration of molecules. The fabrication of patches for transdermal delivery of therapeutic agents is well known to the art.

For administration to the upper (nasal) or lower respiratory tract by inhalation, the therapeutic agents of the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the therapeutic agent may be administered via nose drops, a liquid spray, such as via a plast bottle atomizer or metered-dose inhaler. Typical of atomizers are the Mistometer (Wintrop) and the Medihaler (Riker).

The local delivery of the therapeutic agents of the invention can also be by a variety of techniques which administer the agent at or near the site of disease. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

For topical administration, the therapeutic agents may be formulated as is known in the art for direct application to a target area. Conventional forms for this purpose include wound dressings, coated bandages or other polymer coverings, ointments, creams, lotions, pastes, jellies, sprays, and aerosols, as well as in toothpaste and mouthwash, or by other suitable forms, e.g., via a coated condom. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The active ingredients can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-25% by weight.

When desired, the above-described formulations can be adapted to give sustained release of the active ingredient employed, e.g., by combination with certain hydrophilic polymer matrices, e.g., comprising natural gels, synthetic polymer gels or mixtures thereof.

Drops, such as eye drops or nose drops, may be formulated with an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agent may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; mouthwashes comprising the composition of the present invention in a suitable liquid carrier; and pastes and gels, e.g., toothpastes or gels, comprising the composition of the invention.

The formulations and compositions described herein may also contain other ingredients such as antimicrobial agents, or preservatives. Furthermore, the active ingredients may also be used in combination with other therapeutic agents.

The invention will be further described by the following examples.

### EXAMPLE 1

### Preparation and Characterization of Natriuretic Peptide Encoding Gene Transfer Vectors

Methods

Western Blot Analysis

Samples of equal amounts of protein are denatured by boiling for 5 minutes and resolved by electrophoresis on a 12% SDS-polyacrylamide gel. Transfer of protein to a nitrocellulose membrane is carried out over 3 hours at 4° C. Immunoblotting is performed using a polyclonal rabbit anti-human BNP antibody or polyclonal anti-canine BNP antibody (Phoenix Pharmaceuticals, Mountain View, Calif.) at a dilution of 1:500 in nonfat milk/TBS-T buffer. Following washes, the membrane is subsequently probed with anti-rabbit secondary antibody conjugated to horseradish peroxidase (Amersham Life Sciences, Arlington Heights, Ill.) at a dilution of 1:5000 and developed with chemiluminescence (Supersignal, Pierce, Rockford, Ill.). The membrane is then exposed to X-ray film (Kodak, Rochester, N.Y.) and subsequently developed.

High Performance Gel Permeation Chromatography (HP-GPC)

Natriuretic peptide components of different molecular weight are measured by radioimmunoassay after separation with HP-GPC using a TSK-GEL G 2000 SW column (7.5×600 mm; Toyo Soda, Tokyo), as described in Rodeheffer et al. (1993) and Wei et al. (1993). Columns are eluted with 10 mmol/L trifluoroacetic acid containing 9.2 mol/L sodium chloride and 30% acetonitrile as a solvent at a flow rate of 0.3 mL/min.

cGMP Determinations

Glomeruli are isolated using a modification of the technique of Chaumet-Riffaud et al. (1981). Briefly, after the kidneys are removed and placed in ice-cold Kreb's buffer, pH 7.4, containing 135 mM NaCI, 4.7 mM KCl, 25 mM Na bicarbonate, 1.2 mM K₂HPO₄ 2.5 mM CaCl₂ 0.026 mM Ca disodium versenate, and 10 mM glucose, equilibrated with 95% O₂ and 5% CO₂. The renal cortex is isolated, sliced, minced and centrifuged at 1000 rpm in Kreb's buffer. This mixture is then squeezed through sieves of with pore sizes of 250, 212, and 60 μm sequentially. The glomeruli which are retained on the 60 μm sieve are resuspended in ice-cold Kreb's buffer. The final centrifugation uniformly yields material containing >90% glomeruli and less that 5% tubular cell contamination when examined by light microscopy.

For the measurement of natriuretic peptide dependent cGMP accumulation, aliquots of fresh glomeruli are suspended in 350 ml of buffer A Chaimet-Riffaud et al., 1981 in which 1 mM CaCl₂ is added to glomeruli. Each preparation is preincubated for 10 minutes at 37° C. in a shaking water bath. Incubation is then started in the presence or absence of 3×10⁴ MIBMX, a phosphodiesterase inhibitor with conditioned media. The reaction is terminated at 10 minutes by adding 750 ml ice-cold trichloroacetic acid (TCA), final concentration 6.6%, and centrifugation at 4° C. The pellet is dissolved in 1 N NaOH and assayed for protein by the Lowry method utilizing bovine serum albumin as the standard. The supematant fluid is extracted five times with water saturated ethanol to remove the TCA before being evaporated to dryness under a stream of air and stored at −80° C. until assayed for cGMP content. Cyclic GMP content is determined by dissolving the samples in 50 mM sodium acetate buffer, pH 6.2. 100 ml aliquots are acetylated according to the manufacturer's instructions (New England Nuclear). Averaged results of triplicate determinations are expressed as fmol cGMP accumulated per 10 minute incubation per mg protein. Cyclic GMP is measured by the radioimmunoassay technique of Steiner et al., (1972).

Results

Non-Viral Vectors

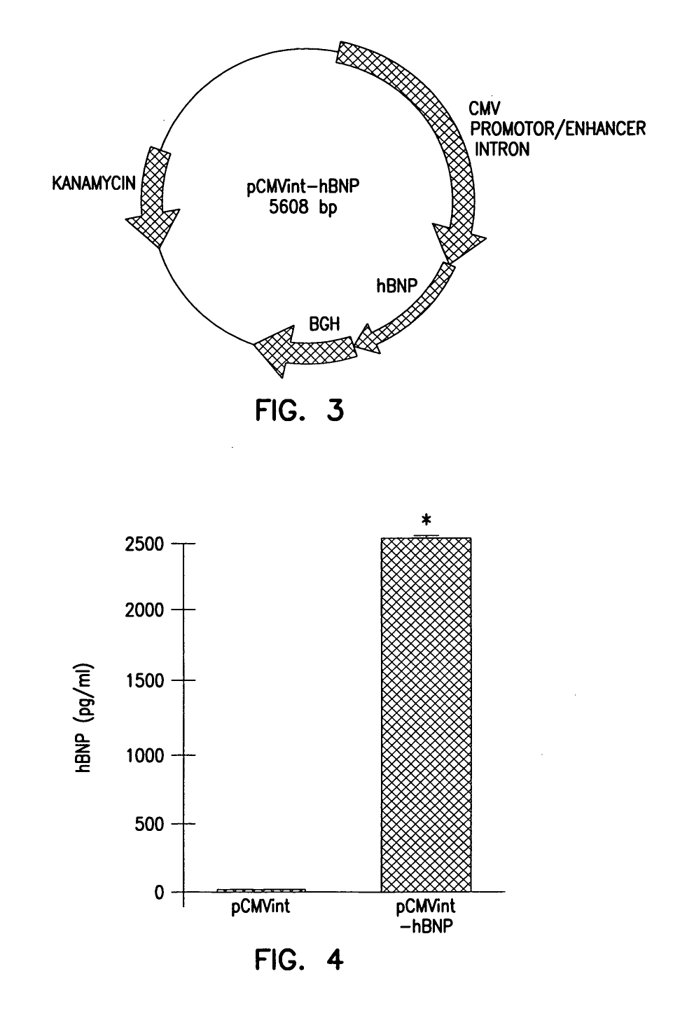

To develop a eukaryotic expression plasmid for human BNP, CDNA encoding for human pre-pro BNP (Seilhamer et al., 1989; obtained from Scios, Inc., Mountain View, Calif.) was amplified from human atrial tissue and cloned into pUC9 at the EcoRI site. This cDNA encodes the 134 amino acid pre-pro form of BNP. The CDNA was excised from this plasmid with EcoRI, treated with the Klenow fragment of DNA polymerase (New England Biolabs, Beverly, Mass.) in the presence of dNTP and cloned into pCMVint at the EcoRV site by blunt ligation with T4 DNA ligase (New England Biolabs, Beverly, Mass.). Untranslated sequences from the cytomegalovirus immediate-early (CMV IE) gene (First exon and intron) were employed to increase expression from CMV IE and heterologous promoters. This enhancement requires the 3′splicing site within the intron and is not dependent on putative enhancers within the intron (Simari et al., 1998; Caplice et al., 1997). Ligation ends of this plasmid (pCMVint-hBNP; FIG. 3) were sequenced. The Limulus amoeba lysate assay is employed to determine the levels of endotoxin in each preparation (preparations were excluded if >5 IU/mg of DNA) (Sigma).

To test the ability of pCMVint-hBNP to express pre-pro BNP from human cells, 293 cells (human embryonic kidney cells) were transfected with pCMVint-hBNP using lipofectamine (Gibco/BRL, Gaithersburg, Md.). The DNA-liposomes were removed following two hours of exposure at 37° C. and replaced with DMEM (GIBCO-BRL, Grand Island, N.Y.) containing 10% fetal calf serum and 1% penicillin/streptomycin. Twenty-four hours later, the media was removed and the cells were washed and the media was replaced with 6 mls of serum-free DMEM for 48 hours at 37° C. Following this incubation, conditioned media was removed, spun at 12,000 rpm for 10 minutes to remove cellular contaminants and frozen in aliquots at −80° C. for later analysis.

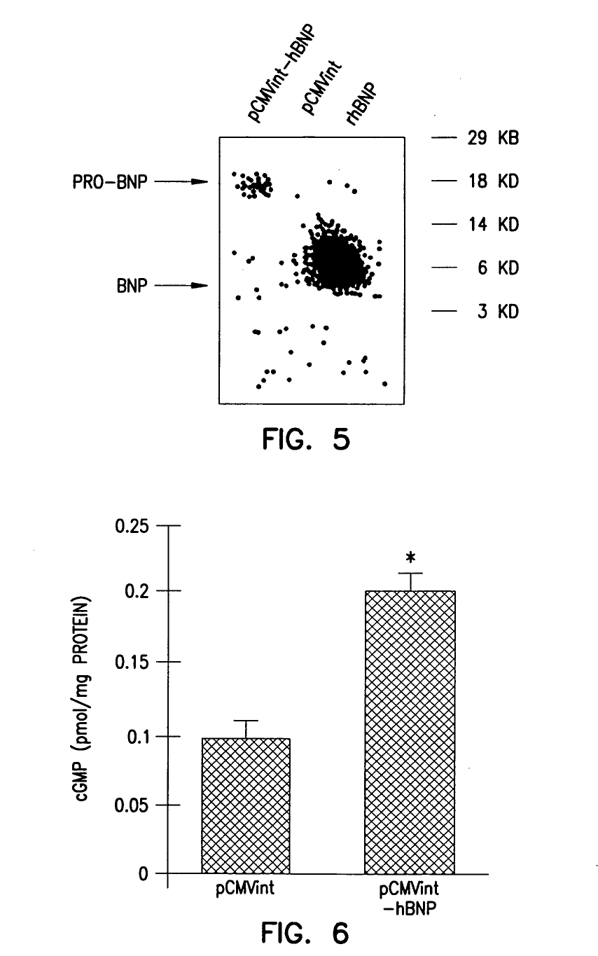

Protein in conditioned media was analyzed using Western blot analysis. The data (FIG. 5) suggests that conditioned media from transfections with pCMVint-hBNP contained an immunoreactive band of a higher molecular weight than recombinant hBNP. This larger form is likely pro BNP but may represent glycosylation of a processed form. Blotting with an antibody to pre-pro forms (Phoenix Pharmaceuticals), high performance gel permeation chromatography, and peptide sequencing (Mayo Peptide Core) are underway to determine more accurately which form this higher MW band represents.

FIG. 4 shows that transfection of pCMVint-hBNP into human cells resulted in >1000-fold increase in expression of BNP, as determined by a radioimmunoassay for BNP (Shionogi Co. Ltd., Osaka, Japan). The ability of these media to stimulate the second messenger, cyclic GMP was also determined in normal canine isolated glomeruli as measured by the radioimmunoassay technique of Steiner et al. (1972). Conditioned media from transfections with pCMVint-hBNP were able to stimulate the production of cGMP compared with control-conditioned media (FIG. 6).

A cDNA encoding pre-pro human BNP was cloned into VR1012 (Vical, Inc., San Diego, Calif.) which contains the CMV IE promoter/enhancer and 5′ untranslated sequences and a bovine growth hormone poly A sequence (VR1012-hBNP). Human embryonic kidney cells were transfected using lipofectamine with VR1012-hBNP or with VR1012. Conditioned media from cells transfected with VR1012-hBNP contained elevated levels of hBNP as detected by radioimmunoassay (RIA) in comparison to media from cells transfected with VR1012 (2475.0±24.1 vs. 4.2±0.3 pg/ml, p<0.05). Western blot analysis of conditioned media using polyclonal antibodies to the pre-pro form and mature forms of BNP demonstrated the presence of a processed from of BNP in media from VR1012-hBNP transfected cells. The ability of the conditioned media to stimulate the second messenger cGMP was assessed via incubation with isolated canine glomeruli. The media from VR1012-hBNP resulted in increased cGMP production relative to VR1012 media (0.40±0.04 vs. 0.20±0.03 pmol/mg protein, p<0.05).

To develop a eukaryotic expression plasmid that expresses canine BNP, a plasmid containing genomic DNA encoding for canine pre-pro BNP (obtained from Scios, Inc., Mountain View, Calif.), which encodes the 134 amino acid pre-pro form of canine BNP, was used as a template in a PCR with primers. The ampli fied sequence was cloned into pCR2.1 (Invitrogen, Carlsbad, Calif.) using T overhangs. The insert was sequenced, and then excised from this plasmid with EcoRI, treated with the Klenow fragment of DNA polymerase (New England Biolabs, Beverly, Mass.) in the presence of dNTP and cloned into pCMVint at the EcoRV site by blunt ligation with T4 DNA ligase (New England Biolabs, Beverly, Mass). Ligation ends of this plasmid (pCMVint-gcBNP; FIG. 7) were sequenced.

cDNA for canine BNP (cBNP) is obtained from RNA from cells transfected with pCMVint-gcBNP using reverse transcriptase-PCR. Briefly, mRNA in 0.5 μg total RNA is transcribed to cDNA in a mixture containing MuLV reverse transcriptase (2.5 μ/ml), 2.5 μM oligo dT primers, 5 mM MgCl₂, 1 mM each of deoxynucleoside triphosphates and 1 U/ml RNase inhibitor in PCR buffer (Perkin Elmer, Foster City, Calif.). Reactions are carried out at 42° C. for 15 minutes followed by MuLV RT inactivation at 99° C. for 5 minutes. PCR samples (20 μl) from each RT mixture are amplified for 35 cycles with Taq polymerase (25 U/ml) using 0.15 μM each of canine-specific BNP oligonucleotide primers. Amplifications are carried out in a thernocycler, 105 seconds at 95° C., 15 seconds at 95° C., 30 seconds at 60° C. and 7 minutes at 72° C. for 35 cycles. The cDNA is cloned into pCR2.1 (Invitrogen, Carlsbad, Calif.) using T overhangs. The insert is sequenced, then excised with EcoRI, treated with the Klenow fragment of DNA polymerase (New England Biolabs, Beverley, Mass.) in the presence of dNTP and cloned into pCMVint at the EcoRV site by blunt ligation with T4 DNA ligase (New England Biolabs, Beverly, Mass.) (pCMVint-cBNP).

To create expression plasmids which express the mature form of cBNP directly, a unique deletion mutant is created using the known sequence of HBNP, which maintains the native leader sequence while removing the pro-enzyme coding sequence. Two long oligos which have 20 bp complementarily are annealed using standard techniques. The oligos each contain a unique 5′ restriction site (XbaI and BgIII) for subsequent cloning. The annealed oligos are extended using T7 DNA polymerase in the presence of dNTP, cleaved with the unique restriction enzymes and cloned into pCMVint at the XbaI and BgIII sites using T4 DNA ligase. The resulting plasmid is referred to as pCMVint-cmBNP (FIG. 10).

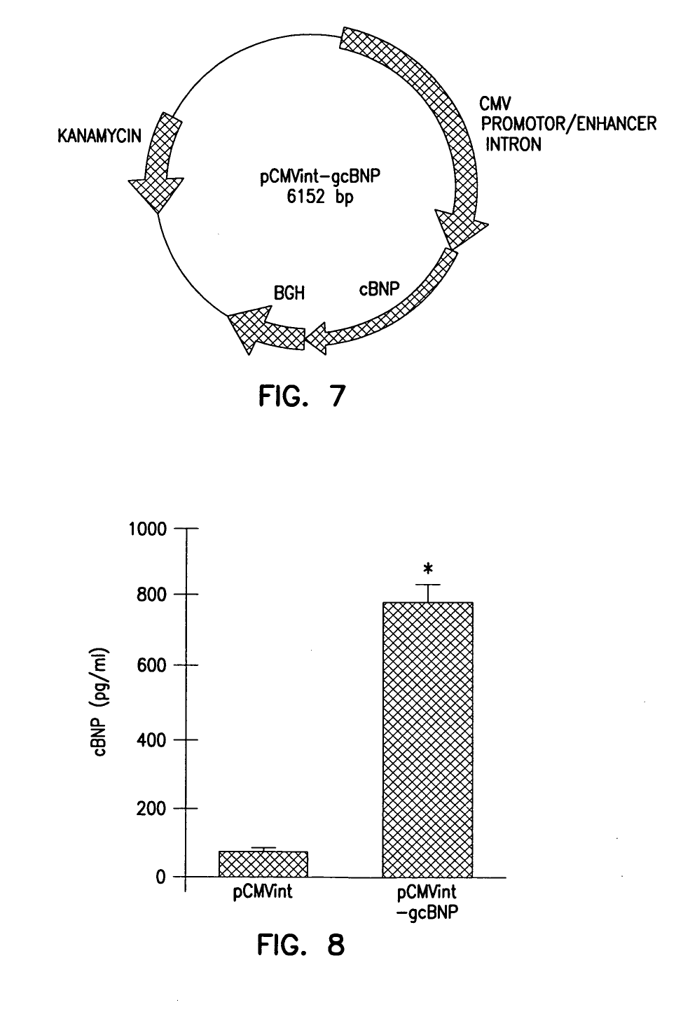

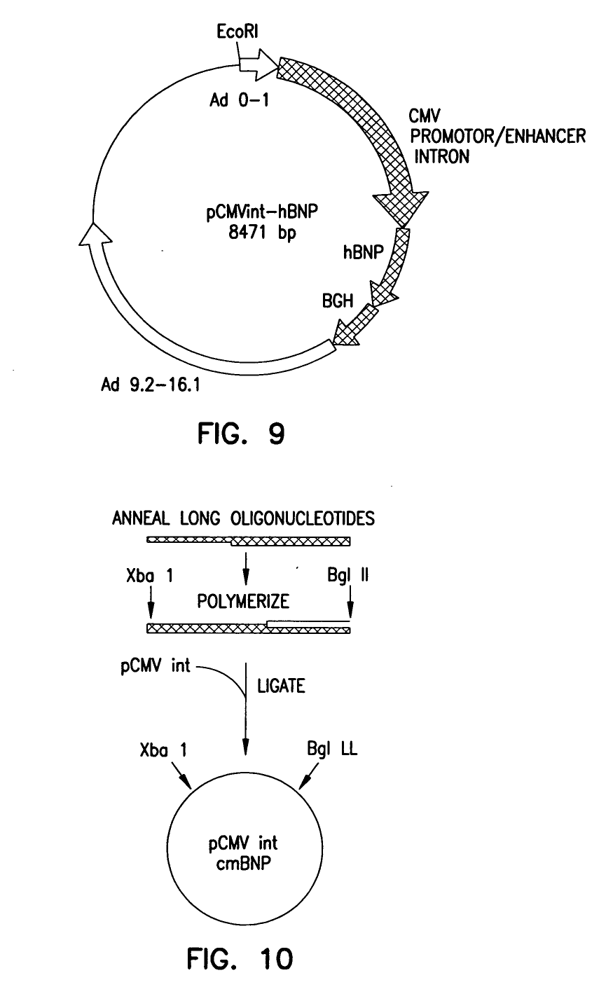

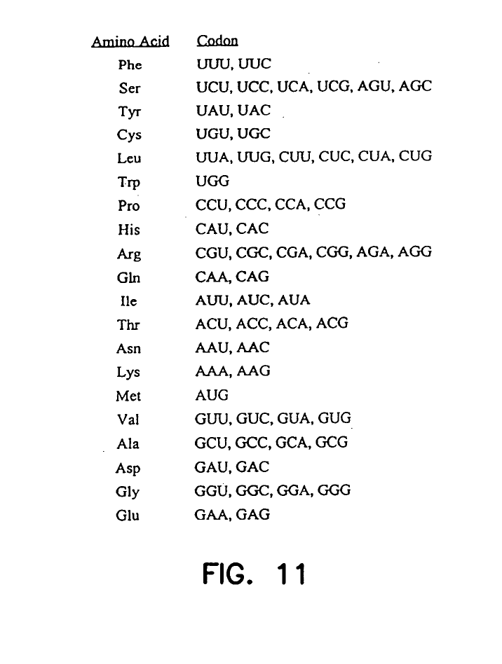

To test the ability of pCMVint-gcBNP to express pre-pro cBNP from human cells, 293 cells (human embryonic kidney cells) were transfected with pCMVint-gcBNP and lipofectamine (Gibco/BRL, Gaithersburg, Md.). As shown in FIG. 8, transfection of pCMVint-gcBNP into human cells resulted in >25 fold increase in expression of BNP.

To determine whether furin processes pro BNP, the furin gene is cloned into a bicistronic plasmid encoding BNP downstream from an internal ribosomal entry site (IRES) from the encephalomyocarditis virus. This plasmid is referred to as pCMVint-cBNP-furin. This plasmid alone, as well as a plasmid expressing cBNP and a plasmid expressing furin, are transfected into cells and the molecular forms of the resulting peptides in conditioned media determined using immunoblotting and HP-GPC.

Adenovirus Vectors

To prepare adenoviral vectors encoding forms of BNP, a shuttle plasmid containing the expression cassette for human BNP (pAdCMV-hBNP) was constructed by cloning the MscI/XmnI fragment of pCMVint-hBNP into the BgIII site of pAdBgIII. pAdBgIII contains the Ad5 sequences 0-1 and 9.2-16.1. This shuttle plasmid is linearized and cotransfected individually with an XbaI/ClaI fragment of Ad5 (sub 360) DNA into 293 cells (human embryonic kidney cells which express E1) to generate replication-deficient recombinant adenoviral vectors expressing hBNP. An E1deleted recombinant adenoviral vector without cDNA insert (Ad-ΔE1) is used as a control for adenoviral infection (Simari et al., 1996). Cotransfection resulted in cytopathic effects in 293 cells.

Crude viral lysates from these transfections is plaque purified and screened for recombinant virus. Cesium chloride purified virus is dialyzed against phosphate buffered saline (PBS), filtered and diluted in 13% glycerol-PBS. Viral titers are determined by standard spectrophotometric methods. Viral stocks are diluted to titers of approximately 1×10¹⁰ pfu/ml. Wild-type virus is excluded using standard techniques.

Other viral vectors constructed include AdCMV-cBNP, which is a vector that expresses pre-pro cBNP from cDNA; AdCMV-cmBNP and AdCMV-cBNP/furin which expresses pre-pro hBNP from cDNA or which expresses mature cBNP, respectively; and AdCMV-gcBNP, which expresses pre-pro cBNP from genomic DNA.

### EXAMPLE 2

### Delivery of BNP In Vivo to Normal Canines

Methods

Plasma and Urine RIAs

Arterial blood for hormone analysis is collected in heparin and EDTA tubes and immediately placed on ice. After centrifugation at 2,500 rpm at 4° C., the plasma is decanted and stored at −80° C. until analysis. Specific plasma radioimmunoassays include canine and human (if necessary) ANP, BNP, CNP, cGMP, renin, and aldosterone. Radioimmunoassays are performed based upon well known methods. Urine for hormone analysis is also collected on ice. Urine samples are analyzed for BNP and cGMP via species-specific radioimmunoassays.

Echocardiographic Analysis

To determine the myocardial effects of local and systemic gene transfer of BNP, a two-dimensional and 2-D guided M-mode echocardiogram (Toshiba, Japan) is performed from the right peristernal window of each dog at baseline and weekly throughout the animal studies. Left ventricular end-diastolic (LVEDd) and end-systolic (LVESd) dimensions are measured from the 2-D guided M-niode tracings. Echocardiograms are performed in the conscious state with the dog unrestrained and standing quietly by a single echocardiographer with over five years of experience performing echocardiography in humans and dogs. Three cardiac cycles are measured and the average of the three measurements recorded. No cycles after a premature or paced premature beat is used for analysis. Echocardiographic formula for the computation of left ventricular ejection fraction is as follows: [(LVEDd²−LVESd²)/LVEDd²].

Analysis of Tissue Samples

To assure that injected DNA remains at the site of injection or infusion, harvested tissue is homogenized and cellular DNA is extracted using standard techniques (Strauss, 1998). PCR of this cellular DNA with primers specific for the unique regulatory sequences of plasmids injected is performed. Sections from each organ are stained with hematoxylin and eosin for evaluation of inflammation at the site of injection and distant sites.

Dosages

A range of single IM (gluteal muscle) doses of from 1-30 mg/animal is employed using a 27 gauge needle. The doses used in adenoviral-based gene transfer range from 10⁷-10¹⁰ pfu (Tripathy et al., 1994).

Results

Local myocardial expression of BNP might achieve higher tissue levels and take advantage of native processing, while skeletal muscle delivery may result in systemic levels that might have greater effects on the renal and vascular systems. To determine whether both routes of administration result in BNP expression, normal dogs are administered a plasmid or recombinant adenovirus which encodes BNP.

Skeletal Muscle-based Gene Transfer in Normal Dogs

Adult mongrel dogs of either sex are used for this protocol. Two weeks prior to starting the protocol, a subcutaneous catheter was placed in mongrel dogs (n=3) under general anesthesia (intravenous 4% Methohexital 1 cc/5 lbs.; maintenance with inhaled Isoflurane) via the femoral artery into the aorta with a distal subcutaneous arterial well permitting serial blood sampling. After appropriate aseptic surgical preparation, a skin incision was made in the left inguinal region and the femoral artery is exposed with blunt dissection. Another skin incision was made on the dog's back in the lumber area. A trocar was passed subcutaneously from the lumbar incision to the groin incision, and the catheter was placed so the end may be inserted into the femoral artery. The port was secured subcutaneously to the lumbar muscle layer with 2-0 silk suture. The arterial end of the catheter was held in situ with 2-0 silk ties. Vicryl was used to close the fascial layers, subcuticular muscle, and the skin. During the surgical procedure, the catheter was filled with a 1:1 solution of heparin and saline. The first day post-op, the catheter was again flushed with this solution to prevent clotting. A weekly flush with Penicillin G Potassium in a 1:1 solution of heparin and saline was employed to maintain patency. Animals were then allowed to recover for two weeks.

On the day prior to IM injection, a 24 hour urine is collected for determination of daily sodium and creatinine excretion permitting assessment of sodium balance and creatinine clearance. Blood is drawn from the arterial port for determination of plasma ANP, BNP, CNP, cGMP, renin, aldosterone, creatinine and sodium together with the measurement of arterial pressure. Following this equilibration, the animals receive an IM injection (hind leg, gluteal muscle) of plasmid (Day 0). Following injection, blood tests are repeated on Days 1, 2, 4 and 7 and weekly thereafter for 42 days. At the same time points, 24 hour urine volumes, creatinine, cGMP and electrolytes and arterial pressure are obtained as well as echocardiography. At 42 days dogs are euthanized and necropsies performed. The muscle at the sites of injection and biopsies of all major organs are removed and flash frozen in liquid nitrogen for further analysis. The hearts are weighed and processed for standard histologic analysis.

Cardiac Muscle-based Gene Transfer in Normal Dogs

Adult mongrel dogs of either sex are used for this protocol. The animals receive an intracoronary infusion of recombinant adenoviral vector (Day 0) Two weeks prior to starting the protocol, a subcutaneous catheter is placed under general anesthesia (intravenous 4% Methohexital 1 cc/5 lbs; maintenance with inhaled Isoflurane) via the femoral artery into the aorta with a distal subcutaneous arterial well permitting serial blood sampling. After appropriate aseptic surgical preparation, a skin incision is made in the left inguinal region and the femoral artery is exposed with blunt dissection. Another skin incision is made on the dog's back in the lumbar area. A trocar is passed subcutaneously from the lumbar incision to the groin incision, and the catheter is placed so the end may be inserted in the femoral artery. The port is secured subcutaneously to the lumbar muscle layer with 2-0 silk suture. The arterial end of the catheter is held in situ with 2-0 silk ties. Vicryl is used to close the fascial layers, subcuticular muscle, and the skin. During the surgical procedure, the catheter is filled with a 1:1 solution of heparin and saline. The first day post-op, the catheter is again flushed with this solution to prevent clotting. A weekly flush is done with Penicillin G Potassium in a 1:1 solution of heparin and saline to maintain patency. Animals are then allowed to recover for two weeks.

One the day prior to gene transfer, a 24 hour urine is collected for determination of daily sodium and creatine excretion permitting assessment of sodium balance and creatinine clearance. Blood is drawn from the arterial port for determination of plasma canine and human (if necessary) ANP, BNP, CNP, cGMP, renin, aldosterone, creatinine and sodium together with the measurement of arterial pressure. Following this equilibraton, the animals receive the intracoronary infusion of adenovirus (Day 0) (Giordano et al., 1996). A midline incision is made in the ventral neck and the right carotid artery is exposed using blunt dissection. An arteriotomy is made and a standard hemostatic sheath is inserted. Through the sheath, a standard 8F angioplasty guiding catheter is placed into the ostium of the left main coronary artery. Under fluoroscopic guidance, an infusion of adenoviral containing solution (4 mls) is infused over 90 seconds. Following delivery, the sheath is removed, the artery ligated and Vicryl is used to close the fascial layers, subcuticular muscle, and the skin.

Blood tests are repeated on Days 1, 2, 4 and 7, and weekly for 6 weeks. At the same time points, 24 hour urine volumes, creatinine, cGMP and electrolytes and arterial pressure are obtained as well as echocardiography. At 42 days, the dogs are euthanized and necropsies performed. The heart is weighed and sectioned for immunohistochemical analysis and biopsies of all major organs is removed and flash frozen in liquid nitrogen for further analysis.

Results An adenoviral vector (ADV-hBNP) encoding the human BNP cDNA for pre-pro BNP was injected into the left ventricular myocardium from the endocardial side in 2 normal dogs using a percutaneous catheter-based technique (Boston Scientific, Natick, Mass.). Each heart was injected at 5 different sites with ADV-BNP (1.4×10⁹ pfu/site) mixed with fluorescent marker beads. Seventy-two hours after gene transfer, animals were euthanized and injection sites were identified by fluorescence. Central venous and coronary sinus human and canine BNP levels were measured at baseline and at 72 hours using specific radioimmunoassays. RT-PCR and immunohistochemistry of injected and control myocardium were performed.

Immunostaining for hBNP showed that BNP was present in myocytes and non-myocytes of treated regions and absent in control areas. hBNP mRNA expression was verified by RT-PCR. At 72 hours, circulating central venous hBNP was 19.4±0.5 pg/mi and coronary sinus hBNP was 208.0±106.1 pg/ml compared to negligible baseline levels (0.6±0.8 pg/ml). Venous canine BNP levels did not change following adenoviral injection (14.1±0.6 at baseline, 16.7 ±2.3 at 3 days). Therefore, catheter-based, adenoviral-mediated gene transfer of human BNP in dogs resulted in local and systemic BNP expression 3 days after infection. These results suggest a new approach for local myocardial and systemic gene-based therapies for cardiovascular diseases.

### EXAMPLE 3

### A Canine ALVD Model

A modified model of pacing-induced ventricular dysfunction is employed to better characterize the temporal changes in local and circulating humoral factors during the progression of experimental heart failure from the initial stage of ventricular systolic failure (ALVD) through the phase of compensation to the terminal phase of overt CHF. Unlike the more conventional model of pacing-induced CHF, this modified model results in progressive ventricular systolic dysfunction with ventricular dilatation and hypertrophy (Stevens et al., 1996).

This model of ALVD is produced by incremental increases in rapid ventricular pacing over a period of a month. Ventricular pacing is initiated first at 180 beats per minute (bpm) and continued at this rate for ten days. This phase mimics human ALVD with early activation of the NPS, a maintenance of sodium balance, suppression of the renin angiotensin aldosterone system and a preserved natriuretic response to intravascular saline volume expansion despite marked ventricular dysfunction (Redfield et al., 1993). The pacing rate is then increased at seven day intervals to rates of 200, 210, 220 and 240 bpm. Neurohumoral function and sodium balance have been characterized at baseline before pacing, in ALVD (180 bpm), in the transition phase (220 bpm) during which the onset of sodium retention is first observed in the absence of a decrease in arterial pressure or increase in circulating angiotensin II and in overt CHF (240 bpm) which is characterized by profound sodium retention with marked activation of angiotensin II. In this model, echocardiography demonstrates a decrease in left ventricular ejection fraction and a progressive increase in left ventricular end diastolic diameter with an increase in left ventricular mass. Left ventricular tissue concentrations of ANP, a marker for hypertrophy, are also increased in overt CHF.

In this model, plasma ANP, BNP and cGMP and urinary cGMP are elevated in ALVD (Stevens et al., 1996). Both ANP and BNP progressively increase during the progression to overt CHF while plasma cGMP plateaus and urinary cGMP decreases. CNP, unlike ANP and BNP, increases minimally and only in overt CHF. Plasma renin activity, aldosterone and ET-1 increase only in overt CHF. Circulating norepinephrine increases prior to the activation of other vasoconstrictive systems during the transition from ALVD to overt CHF. Arterial pressure decreases only in overt CHF while body weight increases during the Transition phase. Urinary sodium excretion is preserved in ALVD despite ventricular dysfunction. Sodium retention is initiated in the Transition phase and was marked in overt CHF. Thus, this model has the cardiorenal and endocrine adaptations similar to that of humans with progressive ventricular dysfunction and represents a model to study therapeutic options to attenuate the progression of this multiorgan disease.

To establish whether overexpression of local or systemic BNP delays the progression of ALVD to overt CHF in a canine model, two groups of dogs are studied in each delivery strategy (cardiac and skeletal muscle). Each group contains ten animals. In the skeletal muscle group, ten receive pCMVint and ten a plasmid-based vector. In the cardiac myocyte group, ten receive Ad-ΔE1 and ten receive a viral vector. Gene transfer is performed on the day prior to initiation of pacing. Prior gene transfer on Day 0, blood measurement of plasma BNP and cGMP and urine for cGMP are obtained. Following injection, blood is drawn on days 2, 7 and 10, and weekly thereafter for a total of 4 weeks.

In a sterile surgical suite, dogs are anesthetized utilizing pentobarbital sodium anesthesia at a dose of 30 mg/kg i.v. for induction and repeated dosing as needed for maintenance of anesthesia. Supplemental oxygenation at 5L/minute is provided with an endotracheal tube utilizing a Harvard respirator (Harvard Apparatus, Millis, Mass.). A programmable cardiac pacemaker (Medtronic, Minneapolis, Minn.) is implanted via a left thoracotomy with a 1-2 cm pericardiotomy. Following the pericardiotomy, the heart is exposed and a screw-in epicardial pacemaker lead is implanted into the right ventricle. The pacemaker lead is then connected to a pulse generator which is implanted subcutaneously in the chest wall. Pacing capture is verified intraoperatively. The pericardium is sutured closed with great care so as not to distort the anatomy of the pericardium. The chest cavity, deep and superficial incisions is closed in layers.

A chronic indwelling femoral arterial catheter for mean arterial pressure monitoring and arterial plasma sampling is placed in each dog during the sterile surgical period. The catheter is implanted into the left femoral artery with the self-sealing silicone rubber septum port tunneled subcutaneously to the left upper hind limb. Arterial line patency is maintained by heparin (1,000 U/ml) flushes which are perfomied twice weekly.

Following the sterile surgical procedure and extubation, each dog spends the next twenty-four hours in a monitored recovery area. A recovery period of fourteen days is allowed following surgical implantation of the pacemaker. Prophylactic antibiotic treatment with 225 mg clindamycin subcutaneously and 400,000 U procaine penicillin G plus 500 mg dihydrostreptomycin intramuscularly are administered preoperatively and on the first two postoperative days. All dogs are fed a controlled sodium diet of 58 mEq sodium per day (Hills I-D) and allowed free access to water.

Progressive left ventricular dysfunction (PLVD) is produced by incremental rapid ventricular pacing with four major periods of study representing the full spectrum of heart failure including asymptomatic left ventricular dysfunction (ALVD), a period of transition between ALVD and severe overt CHF which is a transition period and finally overt CHF. These periods and pacing protocol are as follows:

Baseline: Day 1 prior to pacing

ALVD: Days 1-17 (pacing rate 180 bpm for 10 days followed by 200 bpm for 7 days)

Transition: Days 19-31 (pacing rates 210 bpm for 7 days followed by 220 bpm for 7 days)

Overt CHF: Days 32-38 (pacing rate 240 bpm for 7 days)

Animals are sacrificed on Day 38 following the final assessment.

To assess the myocardial effects of gene transfer, a two-dimensional and 2-D guided M-mode echocardiogram (Toshiba, Japan) is performed from the right peristemal window of each dog at baseline prior to pacing, ALVD (Day 10), transition (Day 31), and in overt CHF (Day 38). Left ventricular end-diastolic (LVEDd) and end-systolic (LVESd) dimensions are measured from the 2-D guided M-mode tracings. Echocardiograms are performed in the conscious state with the dog unrestrained and standing quietly. All images are obtained with the pacemaker deprogrammed for a total imaging period of less than ten minutes. Three cardiac cycles are measured and the average of the three measurements recorded. No cycles after a premature or paced premature beat are used for analysis. Echocardiographic formula for the computation of left ventricular ejection fraction is as follows: [(LVEDd² −LVESd²)/LVEDd²].

To permit characterization of cardiorenal and neurohumoral function, acute experiments are conducted at baseline and ALVD (Day 10), transition (Day 31) and in overt CHF (Day 38). On the night before the acute experiment, animals are fasted, given 300 mg lithium carbonate for assessment of tubular function and allowed access to water. On the day of the acute experiment, dogs are anesthetized with thiopental sodium (15 mg/kg, i.v.) to allow sterile percutaneous placement of a flow-directed balloon tip pulmonary artery catheter placed via an internal jugular vein. The femoral artery catheter is connected to a pressure monitor for on-line measurement of aortic pressure. A urinary bladder catheter is inserted for urine collection. Antibiotic prophylaxis for the acute experiment is provided by intravenous cephazolin (20 mg/kg) 30 minutes prior to the experiment and at completion of the acute protocol. After the dog fully regains consciousness, an equilibration period of sixty minutes is allowed before initiating the acute study. The acute protocol consists of two 45 minute clearances. Hemodynamics are measured and plasma obtained through the arterial port at the midpoint of each collection period. Urine is collected on ice for volume, electrolytes and assays as described herein.

Cardiovascular parameters to be measured during the acute experiment include mean arterial pressure (MAP), right atrial pressure (RAP), pulmonary artery pressure (PAP), pulmonary capillary wedge pressure (PCWP) and cardiac output (CO). Cardiac output is determined by thermodilution (Model 9510-A computer, American Edwards Laboratories, Irvine, CA) measured four times and averaged. Systemic vascular resistance is calculated as [SVR=(mean arterial pressure-right atrial pressure)/cardiac output]. Pulmonary vascular resistance is calculated as [PVR=(pulmonary artery prcssure-pulmonary capillary wedge pressure)/cardiac output]. Mean arterial pressure is assessed via direct measurement from the chronic arterial port. At the completion of the acute study, the pulmonary catheter is removed and pressure applied for homeostasis, followed by a compression Vet Wrap® which is removed four hours after its application.

During each of the acute protocols, insulin and PAH is administered intravenously at the start of the equilibration period as a calculated bolus, followed by a 1 mL/minutc continuous infusion. Glomerular filtration rate is calculated from the clearance of inulin. Renal blood flow is calculated from estimated renal plasma flow (PAH clearance) and hematocrit. Renal vascular resistance is calculated as [RVR=(mean arterial pressure-right atrial pressure)/renal blood flow]. Urinary and plasma insulin concentrations are measured by the anthrone method. Urinary and plasma lithium levels are determined by flame emission spectrophotometry (model 357, Instrumentation Laboratory, Wilmington, Mass.). Lithium clearances are used as an indirect method to calculate distal fractional tubular reabsorption [(clearance of lithium-clearance of sodium)/clearance of lithium]×100.

In the dogs with intracoronary delivery, peptide secretion is determined by methods reported by Wei et al. (1993). A catheter is placed into the coronary sinus (CS) permitting sampling of total heart secretion. Additionally and simultaneously, aortic arch sampling is also obtained. Secretion studies are performed at each time point. The following determination is made: total cardiac secretion will be calculated by the difference in aortic and CS plasma peptide concentration. Prior to tissue harvesting on day 38, after blood sampling from each site, the hearts will be harvested, sectioned, snap frozen with liquid nitrogen, and stored at −80° C. for tissue analysis.

After completion of the protocol, dogs are euthanized and hearts are immediately removed and weighed. All tissue are promptly removed, cut, and placed in liquid nitrogen for freezing prior to storage at −80° C. Urine for hormone analysis is collected on ice as described above. Urine samples are also analyzed for ANP, BNP, CNP, ET-1 and cGMP via species-specific radioimmunoassays.

### EXAMPLE 4

### Humanized Chimeric DNP

To prepare a humanized, mature form of DNP, two oligonucleotides were synthesized as templates for overlap PCR. The first 146mer sense oligonucleotide (SEQ ID NO: 10; 5′GCAGATATCCATGGATCCCCAGACAGCACCTTCCCGGGCGCTCCTGC TCCTGCTCTTCTTGCATCTGGCTTTCCTGGGAGGTCGTTCCCACCCGCT GGGCGAGGTGAAGTACGACCCCTGCTTCGGCCACAAGATCGACCGCA TC 3′) includes an EcoRV site and encodes 30 amino acids of N-terminal hBNp, which includes 26 amino acids of signal peptide and the 4 amino acids following the signal peptide, and 14 amino acids from N-terminal DNP. The second 127mer antisense oligo nucleotide (SEQ ID NO: 11; 5′GAAGATCTTCTTAGGCGCTGGTGCTGGGGGCGTTGGGGCGGGGGTCG CGCAGGCTGGGGCAGCCCAGGTTGCTCACGTGGTTGATGCGGTCGAT CTTGTGGCCGAAGCAGGGGTCGTACTTCACCTC 3′) includes 38 amino acids of DNP, and a BgIII site. These two oligonucleotides were used as templates for overlap PCR to generate the short form chimeric DNP by using BNP-5′(5′-TGCAGATATCCATGGATCCCCAGACAGCAC-3′; SEQ ID NO:12) and DNP-3′(5′-GAAGATCTTCTTAGGCGCTGGTGCTGGGGGCG-3′; SEQ ID NO: 13) as primers.

To prepare a pre-pro form of DNP, the DNA encoding the 106 amino acid fragment of the N-terminus of BNP containing the signal peptide and pro-peptide, was generated by PCR using BNP cDNA as template, and oligonucleotides BNP-5′and hBNPmid (5′-CATCTTGGGGCTTCGTGGTGCCCG; SEQ ID NO:14) as primers. The 175mer antisense oligonucleotide (SEQ ID NO: 15) includes sequences corresponding to 16 amino acids from the C-terminus of HBNP and 38 amino acids of DNP, and a BgIII site. These two fragments were used as templates in overlap PCR with BNP-5′and DNP-3′as primers. The sequence of the 175mer oligonucleotides is GAAGATCTTCTTAGGCGCTGGTGCTGGGGGCGTTGGGGCGGGGGTCG CGCAGGCTGGGGCAGCCCAGGTTGCTCACGTGGTTGATGCGGTCGAT CTTGTGGGCCGAAGCAGGGGTCGTACTTCACCTCCATCTTGGGGCTTC GTGGTGCCCGCAGGGTGTAGAGGACCATTTTGCG.

To efficiently express DNP in heterologous systems, the humanized DNP nucleic acid sequence was cloned downstream of the entire pre-pro sequence of the brain natriuretic peptide (pre-pro BDNP) or the leader sequence of BNP (BDNP) without the prohormone sequences. Transfection of multiple cells (3T3, 293, HepG2) with vectors expressing pre-pro BDNP resulted in nonprocessed (18 kD) forms within cell lysates and a mature form (3 kD) in conditioned media. Transfection with vectors expressing BDNP resulted in mature forms within both cell lysates and conditioned media. Functional studies demonstrated the ability of both forms of BDNP to stimulate cGMP production in HUVECs in an endocrine manner. In addition, both forms completely inhibited serum-stimulated (10% FCS) VSMC proliferation. This stimulation of cGMP and inhibition of proliferation in vascular cells is greater than that seen with BNP expressed in a similar manner. Finally, when exposed to normal porcine arterial rings BDNP caused significant vasorelaxation (>70%) as compared to control media. Thus, expression of pre-pro BDNP results in a processed, mature form of BDNP that is able to stimulate cGMP in vascular cells and has potent antiproliferative and vasoactive properties.

### REFERENCES

Amin, J., Carretero, O., Ito, S. Differential mechanisms of action of ANP and CNP onjuxtamedullary afferent arteriole. JASN. 1995;6:654.
Baumgartner, I., Magner, M., Pieczek, A., Isner, J. Clinical evidence that vascular endothelial growth factor enhance vascular permeability. Circulation. 1997;96(8):I-4 (Abstract).
Bonow, R. New insights into the cardiac natriuretic peptides. Circulation. 1996;93:194601950.
Burnett, J., Jr., Granger, J., Openorth, T. Effects of synthetic atrial natriuretic factor on renal function and renin release. Am J Physiol. 1984;247:F863-F866.
Burnett, J., Jr., Kao, P., Hu, D., Heser, D., Heublen, D., Granger, J., Opgenortb, T., Reeder, G. Atrial natriuretic peptide elevation in congestive heart failure in the human. Science. 1986;231:1145-1147.
Caplice, N., Mueske, C., Kleppe, L., Broze, G., Simari, R. Expression and regulation of tissue factor pathway inhibitor in arteries and vascular smooth muscle cells. Circulation. 1997;96(8):I-663 (Abstract).
Chaumet-Riffaud, P., Oudinet, J.-P., Sraer, J., Lajotte, C., Ardaillou, R. Altered PGE₂ and PGF₂a production by glomeruli and papilla of sodium-depleted and sodium-loaded rats. Am J Physiol. 1981;241:F517-F524.
Chowdhury, J., Grossman, M., Gupta, S., Chowdhury, N., Baker, J., Jr., Wilson, J. Long-term improvement of hypercholesterolemia after ex vivo gene therapy in LDLR-deficient rabbits. Science. 1991;254:1802-1805.
Cody, R., Atlas, S., Laragh, J., Kubo, S., Kovit, A., Ryman, K., Shaknovich, A., Pandolfino, K., Clark, M., Camargo, M., Scarborough, R., Lewicki, J. Atrial natriuretic factor in normal subjects and heart failure patients; plasma levels and renal, hormonal and hemodynamic responses to peptide infusion. J Clin Invest. 1986;78:1362-1374.
Davidson, N., Barr, C., Struthers, A. C-type natriuretic peptide. An endogenous inhibitor of vascular angiotensin-converting enzyrne activity. Hypertension. 1996;93:115-1159.
Davis, H., Whalen, R., Demeneix, B. Direct gene transfer into skeletal muscle in vivo: factors affecting efficiency of transfer and stability of expression. Hum. Gene Ther. 1993;4:151-159.
Davis, H., Demeneix, B., Quantin, B., Coulombe, J., Whalen, R. Plasmid DNA is superior to viral vectors for direct gene transfer into adult mouse skeletal muscle. Hum Gene Ther. 1993;4:733-740.
DeBold, A., Borenstein, H., Veress, A., Sonnenberg, H. A rapid and potent natriuretic response to intravenous injection of atrial myocardial extract in rats. Life Science. 1981;28:89-94.
DeBold, A., Bruneau, B., Kuroski-deBold, M. Mechanical and neuroendocrine regulation of the endocrine heart. Cardiovasc Res. 1996;31:7-18.
Edwards, B., Ackermann, D., Lee, M., Reeder, G., Wold, L., Burnett, J., Jr. Identification of atrial natriuretic factor within ventricular tissue in hamsters and humans with congestive heart failure. J Clin Invest. 15 1988;81:83-86.
Field, L., Veress, A., Steinhelper, M., Cochrane, K., Sonnenberg, H. Kidney function in ANF-transgenic mice. Effect of blood volume expansion. Am J Physiol. 1991;260:RI-R5.
Fraenkel, M., Aldred, G., McDougall, J. Sodium status affects GC-B natriuretic peptide receptor MRNA levels, but not GC-A or C receptor mRNA levels, in the sheep kidney. Clin Sci. 1994;86:517-522.
Gardner, D. Molecular biology of the natriuretic peptides. Trends Cardiovasc Med. 1994;4:159-165.
Giordano, F., Ping, P., McKiman, M., Nozaki, S., Demaria, A., Dillmann, W., Mathieucostello, O., Hammond, H. Intracoronary gene transfer of fibroblast growth factor-5 increases blood flow and contractile function in an ischemic region of the heart. Nature Med. 1996;2:534-539.
Grantham, J., J.C. Burnett, J. BNP: increasing importance in the pathophysiology and diagnosis of congestive heart failure. Cardiovasc Res. 1997;96:388-390.
Grantham, J., Borgeson, D., Burnett, J. BNP: pathophysiological and potential therapeutic roles in acute congestive heart failure. Am J Physiol. 1997;272:R1077-R1083.
Guzman, R., Lemarchand, P., Cystal, R., Epstein, S., Finkel, T. Efficient gene transfer into myocardium by direct injection of adenoviral vectors. Circ Res. 1993;73:1202-1207.
Hunt, P., Yandle, T., Nicholls, M., Richards, A., Espiner, E. The amino-teiminal 5 portion of pro-brain natriuretic peptide (Pro-BNP) circulates in human plasma. Biochem Biophys Res Commun 1995;214(3): 1175-1183.
Isner, J., Walsh, K., Syrnes, J., Pieczek, A., Takeshita, S., Lowry, J., Rossow, S., Rosenfield, K., Weir, L., Brogi, E., Schainfeld, R. Arterial gene therapy for therapeutic angiogenesis in patients with peripheral artery disease. Circulation. 1 996;9 1:2687-2692.
John, S., Krege, J., Oliver, P., Hagaman, J., Hodgin, J., Pang, S., Flynn, T., Smithies, O. Genetic decreases in atrial natriuretic peptide and salt-sensitive hypertension. Science. 1995;267:679-681.
Kass-Eisler, A., Falck-Pedersen, E., Alvira, M., Rivera, J., Buttrick, P., Wittenberg, B., Cipriani, L., Leinwand, L. Quantitative determination of adenovirus-mediated gene delivery to rat cardiac myocytes in vitro and in vivo. Proc Natl Acad Sci USA. 1993;90:11498-11502.
Koller, K., Lowe, D., Bennett, G., Minamino, N., Kangawa, K., Matsuo, H., Goeddel, D. Selective activation of the B-natriuretic peptide receptor by C-type natriuretic peptide (CNP), Science. 1991;252:120-123.
Lee, M., Miller, W., Edwards, B., Burnett, J., Jr. Role of endogenous atrial natriuretic factor in acute congestive heart failure. J Clin Invest. 1989;84:1962-1966.
Lin, H., Parmacek, M., Morle, G., Bolling, S., Leiden, J. Expression of recombinant genes in myocardium in vivo after direct injection of DNA. Circulation. 1990;82:2217-2221.
Lin, K., Chao, J., Chao, L. Human atrial natriuretic peptide gene delivery reduced blood pressure in hypertensive rats. Hypertension. 1995;26(1):847-853.
Lopez, M., Wong, S., Kishinoto, I., Dubois, S., Mach, V., Friesen, J., Garbers, D., Beuve, A. Salt-resistant hypertension in mice lacking the guanylyl cyclase-A receptor for atrial natriuretic peptide. Nature. 1995;378:65-68.
Luchner, A., Stevens, T., Borgeson, D., Redfield, M., Bailey, J., Sandberg, S., Heublein, D., Burnett, J. Angiotensin II in the evolution of experimental heart failure. Hypertension. 1996;28 :472-477.
Manthorpe, M., Cornepert-Jansen, F., Hartikka, J., Felgner, J., Rundell, A., Margalith, M., Dwarki, V. Gene therapy by intramuscular injection of plasmid DNA: studies on firefly luciferase gene expression in mice. Hum Gene Ther. 1993;4:419-431.
Marcus, L., Hart, D., Packer, M., Yushak, M., Medina, N., Danziger, R., Heitjan, D., Katz, S. Hemodynamic and renal excretory effects of human brian natriuretic peptidc infusion in patients with congestive heart failure: A double-blind, placcbo-controlled, randomized crossover trial. Circulation 1996;94:3184-3189.
Mattingly, M., Brandt, R., Heublein, D., Wei, C., Nir, A., Burnett, J., Jr. Presence of C-type natriuretic peptide in human kidney and unne. Kidney Int. 1994;46:744-747.
McDonagh, T. Symptomatic and asymptomatic left-ventricular systolic dysfunction in an urban population. Lancet, 1997;350.
Moore, D. Gene synthesis assembly of target sequences using mutually priming long oligonucleotides. In: Ausubel F, ed. Current Protocols in Molecular Biology. New York: John Wiley and Sons, Inc.; 1998:8.2.13.
Morishita, R., Gibbons, G., Pratt, R., Tomita, N., Kaneda, Y., Ogihara, T., Dzau, V. Autocrine and paracrine effects of atrial natriuretic peptide gene transfer on vascular smooth muscle and endothelial cellular growth. J Clin Invest. 1994;94(2):824-829.
Mukoyama, M., Nakao, K., Hosoda, K., Suga, S., Saito, Y., Ogawa, Y., Shirakami, G., Jougasaki, M., Obata, K., Yasue, H., Kambayashi, Y., lnouye, K., Imura, H. Brain natriuretic peptide as a novel cardiac hormone in humans; evidence for an exquisite dual natriurctic peptide system, atrial natriuretic peptide and brain natriuretic peptide. J Clin Invest. 1991;87:1402-1412.
Nabel, E., Plautz, G., Boyce, F., Stanley, J., Nabel, G. Recombinant gene expression in vivo within endothelial cells of the arterial wall. Science. 1989;244: 1342-1344.
Niu, H., Zimmermann, E., Simari, R., Chistman, G. Nonviral vector mediated thymidine kinase gene transfer and ganciclovir treatment in leiomyoma cells. Obstet Gynecol.: (in press).
Ogawa, Y., Itoh, H., Tamura, N., Suga, S., Yoshimasa, T., Uchira, M., Matsuda, S., Shiono, S., Nishimoto, H., Nakao, K. Molecular cloning of the complementary DNA and gene that encode mouse brain natriuretic peptide and generation of transgenic mice that overexpress the brain natriuretic peptide gene. J Clin Invest. 1994;93(5):191 l-1921.
Ohno, T., Gordon, D., San, H., Pompili, V., Imperiale, M., Nabel, G., Nabel, E.

Gene therapy for vascular smooth muscle cell proliferation after arterial injury. Science. 1994;265 :781-784.
Osborn, W., Ramesh, N., Lau, S., Clowes, M., Dale, D., Clowes, A. Gene therapy for long-term expression of erythropoietin in rats Proc Natl Acad Sci, USA. 1995;92:8055-8058.
Perrella, M., Schwab, T., O'Murchu, B., Redfield, M., Wei, C.M., Edwards, B., Bumet, J. Cardiac atrial natriuretic factor during evolution of congestive heart failure. Am J Physiol. 1992;262:H1248-H1255.
Pfeffer, M., Braunwald, E., Moye, L., Basta, L., Brown, E., Jr., Cuddy, T., Davis, B., Geltman, E., Goldman, S., Flaker, G. Effect of captopril on mortality and morbidity in patients with left ventricular dysfunction after myocardial infarction. Results of the survival and ventricular enlargement trial. N Eng J Med. 1992;237:669-677.
Redfield, M., Aarhus, L., Wright, R., Burnett, J. Cardiorenal and neurohumoral function in a canine model of early left ventricular dysfunction. Circulation. 1993;87:2016-2022.
Rehemtulla, A., Kaufman, R. Preferred sequence requirements for cleavage of pro-von Willebrand factor by propeptide-processing enzymes. Blood. 1991 ;79(9):2349-2355.
Rehemtulla, A., Dorner, A., Kaufman, R. Regulation of PACE propeptide-processing activity: requirement for a post-endoplasmic reticulum compartment and autoproteolytic activation. Proc Natl Acad Sci, USA. 1992;89:8235-8239.
Rodeheffer, R., Naruse, M., Atkinson, J., Naruse, K., Burnett, J., Jr., Merrill, W., First, W., Demura, H., Inagami, T. Molecular forms of atrial natriuretic factor in normal and failing human myocardium. Circulation. 1993;88:364-371.
Rodman, D., San Simari, R., Stephan, D., Tanner, F., Yang, Z., Nabel, G., Nabel, E. In vivo gene delivery to the pulmonary circulation in rats. Transgene distribution and vascular inflammatory response. Am J Respir Cell Mol Biol. 1997; 16:640.
Sawada, Y., Suda, M., Yokoyama, H., Kanda, T., Sakamaki, T., Tanaka, S., Nagai, R., Abe, S., Takeuchi, T. Co-elevation of brain natriuretic peptide and proprotein-processing endoprotease furin after myocardial infarction in rats. FEBS Lett. 1997a;400:177-182.
Sawada, Y., Suda, M., Yokoyama, H., Kanda, T., Sakamaki, T., Tanaka, S., Nagai, R., Abe, S., Takeuchi, T. Stretch induced hypertrophic growth of cardiocytes and processing of brain-type natriuretic peptide are controlled by proprotein-processing endoprotease furin. J Biol Chem. 1997b;272(33):20545-20554.
Schirger, J. A., Heublein, D., Chon, H., Lisy, O., Jougasaki, M., Wennberg, P., Bumett Jr. J.C. Presence of Dendroaspis natriuretic peptide-like immuno-reactivites in human plasma and its increase during human heart failure. Mayo Clin. Proc. 1999, 74:126-130.
Schocken, D., Arrieta, M., Leaverton, P., Ross, E. Prevalence and mortality rate of congestive heart failure in the United States. J Am Coll Cardiol. 1992;20(2):301-306.
Seilhamer, J., Arfsten, A., Miller, J. Human and canine gene homologs of porcine brain natriuretic peptide. Biophys Res Commun. 1989;165:650-658.
Simari, R.D., San, H., Nabel, E.G. Gene transfer to arteries. Current Protocols in Human Genetics. Brooklyn: John Wiley and Sons, Inc.; 1996:13.1.1-13.1.10.
Simari, R., San, H., Rekhter, M., Ohno, T., Gordon, D., Nabel, G., Nabel, E. Regulation of cellular proliferation and intimal formation following balloon injury in atherosclerotic rabbit arteries. J Clin invest. 1996;98:225-235.
Simari, Yang, Z-Y., Ling, X., Stephan D., Perkins, No., Nabel, G., Nabel, E. Requirements for enhanced transgene expression by untranslated sequences from the human cytomegalovirus immediate-early gene. Mol. Med. 1998;4:700-706.
SOLVD investigators. Effect of enalapril on survival in patients with reduced left ventricular ejection fractions and congestive heart failure. N Eng J Med. 1991;325:293.
Steiner, A., Parker, C., Kipnis, D. Radioimmunoassay for cyclic nucleotides. I, Preparation of antibodies and iodinated cyclic nucleotides. J Biol Chem 1972;247:1106-1113.
Steinhelper, M., Cochrane, K., Field, L. Hypotension in transgenic mice expressing atrial natriuretic factor fusion genes. Hypertension 1990;16:301-307.
Stephan, D.S., Yang, Z.Y., San, H., Simari, R.D., Wheeler, C.J., Feigner, P.L., Gordon, D., Nabel, G.J., Nabel, E.G. A new cationic liposome DNA complex enhances the efficiency of arterial gene transfer in vivo. Hum Gene Ther. 1996;7:1803-1812.
Stevens, T., Borgeson, D., Luchner, A., Wennberg, P., Redfield, M., Burnett, J., Jr. A modified model of tachycardia-induced cardiomyopathy; Insights into humoral and renal adaptations. In: Spinale F, ed. Pathophysiology of Tachycardia-Induced Heart Failure. New York: Futura Publishing Company, Inc.; 1996:133-151.
Stevens, T., Rasmussen, T., Wei, C.-M., Kinoshita, M., Matsuda, Y., Burnett, J., Jr. Renal role of the endogenous natriuretic peptide system in acute congestive heart failure. J Card Fail. 1996;2(2):119-125.
Stevens, T., Burnett, J., Kinoshita, M., Matsuda, Y., Redfield, M. A ftinctional role for endogenous atrial natriuretic peptide in a canine model of early left ventricular dysfunction. J Clin Invest. 1995;85:1101-1108.
Stevens, T., Wei, C., Aarhus, L., Heublein, D., Kinoshita, M., Matsuda, Y., Burnett, J. Modulation of exogenous and endogenous atrial natriuretic peptide by a receptor antagonist. Hypertension. 1994;23:613-618.
Stingo, A., Clavell, A., Heublein, D., Wei, C., Pittelkow, M., Burnett, J., Jr. Presence of C-type natriuretic peptide in cultured human endothelial cells and plasma. Am J Physiol 1992;263:H1318-HI321.
Stingo, A., Clavell, A., Aarhus, L., Burnett, J., Jr. Cardiovascular and renal actions of C-type natriuretic peptide. Am J physiol. 1992;262:H308-H312.
Strauss, W. Preparation of genomic DNA from mammalian tissue. In: Ausubel F, ed Current Protocols in Molecular Biology. New York: John Wiley and Sons, Inc.; 1998:2.2.1-2.2.3.
Svensson, E., Tripathy, S., Leiden, J. Muscle-based gene therapy: realistic possibilities for the future. Molecular Medicine Today. 1996;2(4):166-172.
Takahashi, T., Allen, P., Izumo, S. Expression of A-, B-, and C-type natriuretic peptide genes in failing and developing human ventricles. Circ Res. 15 1992;71:9-17.
Tateyama, H., Hino, J., Minamino, N., Kangawa, K., Minamino, T., Sakai, K., Ogihara, T., Matsuo, H. Concentrations and molecular forms of human brain natriuretic peptide in plasma. Biochem Biophys Res Commun. 1992; 1 85(2):760-767.
Tripathy, S., Svensson, E., Black, H., Goldwasser, E., Margalith, M., Hobart, P., Leiden, J. Long-term expression of erythropoietin in the systemic circulation of mice after intramuscular injection of a plasmid DNA vector. Proc Natl Acad Sci, USA. 1996a;93:10876-10880.
Tripathy, S., Black, H., Goldwasser, E., Leiden, J. Immune responses to transgene-encoded proteins limit the stability of gene expression after injection of replication-defective adenovirus vectors. Nature Med. 1996b;2(5):545-550.
Tripathy, S., Goldwasser, E., Lu, M.-M. Barr, E., Leiden, J. Stable delivery of physiologic levels of recombinant erythropoietin to the systemic circulation by intramuscular injection of replication-defective adenovirus. Proc Natl Acad Sci, USA. 1994;91:11557-11561.
Tsunimi, Y., Takeshita, S., Chen, D., Kearney, M., Rossow, S., Passeri, J., Horowitz, J., Symes, J., Isner, J. Direct intramuscular gene transfer of naked DNA encoding vascular endothelial growth factor augments collateral development and tissue perfusion. Circulation. 1996;94:3281-3290.
Ueno, H., Haruno, A., Morisaki, N., Furuya, M., Kangawa, K., Takeshita, A., Saito, Y. Local expression of C-type natriuretic peptide markedly suppresses neointimal formation in rat injured arteries through an autocrine/paracrine loop. Circulation. 1997;96:2272-2279.
Wei, C.-M., Heublein, D., Perrella, M., Lerman, A., Rodeheffer, R., McGreagor, C., Edwards, W., Schaff, H., Burnett, J. Natriuretic peptide system in human heart failure. Circulation. 1993;88:1004-1009.
Welling, T., Davidson, B., Zelenock, J., Stanley, J., Gordon, D., Roessler, B., Messina, L. Systemic delivery of the interleukin-l receptor antagonist protein using a new strategy of direct adenoviral-mediated gene transfer to skeletal muscle capillary endothelium in the isolated rat hind limb. Hum Gene Ther. 1996;7:1795-1802.
Wolff, J., Malone, R., Williams, P., Chong, W., Acsadi, G., Jani, A., Felgner, P. Direct gene transfer into mouse muscle in vivo. Science. 1990;247: 1465-1468.
Yasue, H., Yoshimura, M., Sumida, H., Kikuta, K., Kigiyama, K., Yougasaki, M., Ogawa, H., Okumura, K., Mukoyama, M., Nakao, K. Localization and mechanism of secretion of B-type natriuretic peptide in comparison with those of A-type natriuretic peptide in normal subjects and patients with heart failure. Circulation. 1994;90:195-203.

All publications, patents and patent applications are incorporated herein by reference. While in the foregoing specification this invention has been described in relation to certain preferred embodiments thereof and many details have been set forth for purposes of illustration, it will be apparent to those skilled in the art. that the invention is susceptible to additional embodiments and that certain of the detailed herein may be varied considerably without departing from the basic principles of the invention.

1-4. (canceled)

5. A method to inhibit or prevent hypertension in mammal, comprising: administering to the mammal an effective amount of a composition comprising a nucleic acid molecule comprising a nucleic acid segment encoding natriuretic peptide or a chimera thereof in a delivery vehicle.

6-16. (canceled)

17. The method of claims wherein the delivery vehicle is a recombinant adenovirus.

18. The method of claim 17 wherein the nucleic acid molecule is an adenovirus vector.

19. The method of claims 5 wherein the composition is administered via intramyocardial injection.

20. The method of claim 5, wherein the administration is local.

21. The method of claims 5 wherein the administration is systemic.

22. The method of claims 5 wherein the delivery vehicle is a liposome or aqueous media.

23. The method of claim 22 wherein the nucleic acid molecule is a plasmid.

24. (canceled)

25. The method of claims 5 wherein the nucleic acid molecule encodes human brain natriuretic peptide.

26. The method of claims 5 wherein the nucleic acid molecule encodes canine brain natriuretic peptide.

27. The method of claims 5 wherein the composition is administered to skeletal muscle.

28. The method of claim 5 wherein the composition is administered to cardiac muscle.

29. The method of claims 5 wherein the composition is administered via a catheter.

30. The method of claims 5 wherein the nucleic acid molecule further comprises a promoter.

31-46. (canceled)


SEQUENCE LISTING (18 sequences)

SEQ ID NO: 1 - DNA, 1922 bp, Homo sapiens
ctgtgagatcaccccgtgctcccagcgctcacgtcggtcctcggaaagccggggtcctccctgccttttccagcaacggtggggtggggaggcaggaagaaagcgccaacctaggaccccggagatttgcagcaaaggaagaagcgggagacgggcacttgtctgtgtctccagcgcgttcctgccccccgccgacccggcccatttctatacaaggtcgctctgcccggtctccacctcccacgtgcaggccgcggaggggctcattcccgggccctgatctcagaggcccggaatgtggctgataaatcagagactagacctgcatggcaggcaggcccgacactcagctccaggataaaaggccacggtgtcccgaggagccaggaggagcaccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggtgagagcggagggcagctcagggggattggacagcagcaatgaaagggtcctcacctgctgtcccaagaggccctcatctttcctttggaattagtgataaaggaatcagaaaatggagagactgggtgccctgaccctgtacccaaggcagtcggttcacttgggtgccatgaagggctggtgagccaggggtgggtccctgaggcttggacgcccccattcattgcaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaaggtaagcaccccctgccaccccggccgccttcccccattccagtgtgtgacactgttagagtcactttggggtttgttgtctctgggaaccacactctttgagaaaaggtcacctggacatcgcttcctcttgttaacagccttcagggccaaggggtgcctttgtggaattagtaaatgtgggcttatttcattaccatgcccacaataccttctccccacctcctacttcttatcaaaggggcagaatctcctttgggggtctgtttatcatttggcagccccccagtggtgcagaaagagaaccaaacatttcctcctggtttcctctaaactgtctatagtctcaaaggcagagagcaggatcaccagagcaatgataatccccaatttacagatgaggaaactgaggctcagagagttgcattaagcctcaaacgtctgatgactaacagggtggtgggtggcacacgatgaggtaagctcagcccctgcctccatctcccaccctaaccatcatcaccctctctctttccctgacagtgctgaggcggcattaagaggaagtcctggctgcagacacctgcttctgattccacaaggggctttttcctcaaccctgtggccgcctttgaagtgactcattttttttaatgtatttatgtatttatttgattgttttatataagatggtttcttacctttgagcacaaaatttccacggtgaaataaagtcaacattataagctttatcttttgaaactgatttgtcttggcgcattaaaaataatccctcatttcaaagaa

SEQ ID NO: 2 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 3 - protein, 32 aa, Canis sp.
SPKMMHKSGCFGRRLDRIGSLSGLGCNVLRKY

SEQ ID NO: 4 - DNA, 1803 bp, Canis sp.
cgatcagggatgttggggcggaggaaacggagggaaggagggagcggaggaggcccgaggactgttggtgtccccctcctgcccttttggggccaggcccacttctatacaaggcctgctctccagcctccaccccggcgggtatggtgcaggcgcggaggggcgcattcccccgccctgagctcagcggccggaatgcggccgataaatcagagataaccccaggcgcgggataagggataaaaagcccccgttgccgcgggatccaggagagcacccgcgccccaagcggtgacactcgaccccggtcgcagcgcagcagctcagcagccggacgtctctttccccacttctctccagcgacatggagccctgcgcagcgctgccccgggccctcctgctcctcctgttcttgcacctgtcgccactcggaggccgcccccacccgctgggcggccgcagccccgcctcggaagcctcggaagcctcagaagcctcggggttgtgggccgtgcaggtgagcgctcagcctgcctgaaggccgcggcgggtggcagcaggtcacgggggcttagccactgtcccaagtcctcagtctcccttgggaattagtgataagggaatcagaaagtgacgagattgggtgccaggactccatacccaaggcggcggcttcacttgggtgcaagggtggttccgccccggcgtgggttcctgaggctcaggccgtccattgcaggagctgctgggccgtctgaaggacgcagtttcagagctgcaggcagagcagttggccctggaacccctgcaccggagccacagccccgcagaagccccggaggccggaggaacgccccgtggggtccttgcaccccatgacagtgtcctccaggccctgagaagactacgcagccccaagatgatgcacaagtcagggtgctttggccggaggctggaccggatcggctccctcagtggcctgggctgcaatggtaagccgcctccctgccgccttggctccccctccccagccccctgggttcgacccttggaaccccttctgggtttgttgtctcgggggatcacactctgaggaaaggacatctggacatcgctccttcttgctgacagtcctaagggccaaggagtacgtttctggaaatactacgtgtggacatcgttgtccagggtccctacccacctcctagccccctcctgcctctcgcacccaagggcagaatcatcttaggatggaatcagtcgttgtctggaagcatctccttggagcagaaagagtcctaaacatcgtcctcgtagctctctctgtctgtctgtagccacgaaggcagaggtcagggtcaccagggcagtgatgattcccagttaacagaggaggagactgaggtctagagagatggattattccaaagcctcaaacatccagatcggctgagggtggggttggtggcagggatggctcctgggcttgggaagctcggatcctgcctcagtctcccacctgacgccatcatccccctctctctcctcccacagtgctgagaaagtattaaggaggaagtcccgactgcccacatctgcattggattcttcagcagcccctgagccccttggaagcagatcttatttattcgtatttatttatttatttatttcgattgttttatataagatgatcctgacgcccgagcacggattttccacggtgaaataaagtcaaccttagagcttcttttgaaaccgatttgtccctgtgcattaaaagtaacacatcatttaaaaaaa

SEQ ID NO: 5 - protein, 4 aa, Artificial Sequence
  consensus sequence
RXXR

SEQ ID NO: 6 - DNA, 330 bp, Homo sapiens
tcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaa

SEQ ID NO: 7 - protein, 109 aa, Homo sapiens
SHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 8 - DNA, 99 bp, Homo sapiens
agccccaagatggtgcaagggtctggctgctttgggaggaagatggaccggatcagctcctccagtggcctgggctgcaaagtgctgaggcggcattaa

SEQ ID NO: 9 - protein, 32 aa, Homo sapiens
SPKMVQGSGCFGRKMDRISSSSGLGCKVLRRH

SEQ ID NO: 10 - DNA, 145 bp, Artificial Sequence
  primer
gcagatatccatggatccccagacagcaccttcccgggcgctcctgctcctgctcttcttgcatctggctttcctgggaggtcgttcccacccgctgggcgaggtgaagtacgacccctgcttcggccacaagatcgaccgcatc

SEQ ID NO: 11 - DNA, 127 bp, Artificial Sequence
  primer
gaagatcttcttaggcgctggtgctgggggcgttggggcgggggtcgcgcaggctggggcagcccaggttgctcacgtggttgatgcggtcgatcttgtggccgaagcaggggtcgtacttcacctc

SEQ ID NO: 12 - DNA, 30 bp, Artificial Sequence
  primer
tgcagatatccatggatccccagacagcac

SEQ ID NO: 13 - DNA, 32 bp, Artificial Sequence
  primer
gaagatcttcttaggcgctggtgctgggggcg

SEQ ID NO: 14 - DNA, 24 bp, Homo sapiens
catcttggggcttcgtggtgcccg

SEQ ID NO: 15 - DNA, 176 bp, Artificial Sequence
  primer
gaagatcttcttaggcgctggtgctgggggcgttggggcgggggtcgcgcaggctggggcagcccaggttgctcacgtggttgatgcggtcgatcttgtgggccgaagcaggggtcgtacttcacctccatcttggggcttcgtggtgcccgcagggtgtagaggaccattttgcg

SEQ ID NO: 16 - protein, 28 aa, Homo sapiens
SLRRSSCFGGRMDRIGAQSGLGCNSFRY

SEQ ID NO: 17 - protein, 22 aa, Homo sapiens
GLSKGCFGLKLDRIGSMSGLGC

SEQ ID NO: 18 - protein, 38 aa, Dendroaspis angusticeps
EVKYDPCFGHKIDRINHVSNLGCPSLRDPRPNAPSTSA